(19) United States
(12) Patent Application Publication
(10) Pub. No.: US 2009/0298866 A1
(43) Pub. Date: Dec. 3, 2009

(54) Indole Compounds

(76) Inventors: Adrian Hall, Essex (GB); David Nigel Hurst, Essex (GB); Tiziana Scoccitti, Essex (GB); Pamela Joan Theobald, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/373,315

(22) PCT Filed: Jul. 9, 2007
(86) PCT No.: PCT/EP07/56942

§ 371 Date: Jan. 12, 2009

(30) Foreign Application Priority Data
Jul. 14, 2006 (GB) 0614070.1

(51) Int. Cl. A61K 31/437; C07D 277/28; C07D 277/24; C07D 417/14; C07D 471/04; A61K 31/427; A61K 31/44; A61P 37/04

(52) U.S. Cl. 514/303; 548/192; 548/194; 546/270.7; 546/118; 514/370; 514/371; 514/333

(57) ABSTRACT

The present application relates to compounds of formula (I) or a pharmaceutically acceptable derivative thereof: formula (I) wherein X, R¹, R², and R³ are as defined in the specification, a process for the preparation of such compounds, pharmaceutical compositions comprising such compounds and the use of such compounds in medicine.

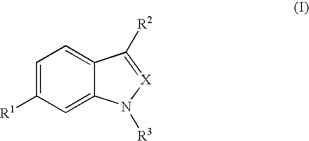

This invention relates to indole compounds, to processes for their preparation, to pharmaceutical compositions containing them and to their use in medicine, in particular their use in the treatment of conditions mediated by the action of PGE₂ at the EP₁ receptor.

The EP₁ receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion. We have now found a novel group of compounds which bind with high affinity to the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S Narumiya et al, Physiological Reviews 1999, 79(4), 1193-126. An article from The British Journal of Pharmacology, 1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. Thus, selective prostaglandin ligands, agonists or antagonists, depending on which prostaglandin E receptor subtype is being considered, have anti-inflammatory, antipyretic and analgesic properties similar to a conventional non-steroidal anti-inflammatory drug, and in addition, inhibit hormone-induced uterine contractions and have anti-cancer effects. These compounds have a diminished ability to induce some of the mechanism-based side effects of NSAIDs which are indiscriminate cyclooxygenase inhibitors. In particular, the compounds have a reduced potential for gastrointestinal toxicity, a reduced potential for renal side effects, a reduced effect on bleeding times and a lessened ability to induce asthma attacks in aspirin-sensitive asthmatic subjects. Moreover, by sparing potentially beneficial prostaglandin pathways, these agents may have enhanced efficacy over NSAIDS and/or COX-2 inhibitors.

In The American Physiological Society (1994, 267, R289-R-294), studies suggest that PGE₂-induced hyperthermia in the rat is mediated predominantly through the EP₁ receptor.

WO 96/06822 (7 Mar. 1996), WO 96/11902 (25 Apr. 1996), EP 752421-A1 (8 Jan. 1997), WO 01/19814 (22 Mar. 2001), WO 03/084917 (16 Oct. 2003), WO 03/101959 (11 Dec. 2003), WO 2004/039753 (13 May 2004), WO 2004/083185 (30 Sep. 2004), WO 2005/037786 (28 Apr. 2005), WO 2005/037793 (28 Apr. 2005), WO 2005/037794 (28 Apr. 2005), WO 2005/040128 (6 May 2005), WO 2005/054191 (16 Jun. 2005), WO2005/108369 (17 Nov. 2005), WO 2006/066968 (29 Jun. 2006), WO 2006/114272 (2 Nov. 2006), WO 2006/114274 (2 Nov. 2006) and WO 2006/114313 (2 Nov. 2006) disclose compounds as being useful in the treatment of prostaglandin mediated diseases.

P. Lacombe et al, (220th National Meeting of The American Chemical Society, Washington D.C., USA, 20-24 August, 2000) disclosed 2,3-diarylthiophenes as ligands for the human EP₁ prostanoid receptor. Y. Ducharme et al, (18^{th} International Symposium on Medicinal Chemistry; Copenhagen, Denmark and Malmo, Sweden; 15^{th}-19^{th} Aug. 2004) disclosed 2,3-diarylthiophenes as EP₁ receptor antagonists. Y. Ducharme et al, Biorg. Med. Chem. Lett., 2005, 15(4): 1155 also discloses 2,3-diarylthiophenes as selective EP₁ receptor antagonists.

S. C. McKeown et al, Bioorg. Med. Chem. Lett., 2007, 17, 1750; A. Hall et al, Bioorg. Med. Chem. Lett., 2007, 17, 1200; A. Hall et al, Bioorg. Med. Chem. Lett., 2007, 17, 916; A. Hall et al., Bioorg. Med. Chem. Lett., 2007, 17, 732; G. M. P. Giblin et al., Bioorg. Med. Chem. Lett., 2007, 17, 385-389; S. C. McKeown et al, Bioorg. Med. Chem. Lett., 2006, 16 (18), 4767-4771;” A. Hall et al., Bioorg. Med. Chem. Lett., 2006, 16 (14), 3657-3662; and A. Hall et al., Bioorg. Med. Chem. Lett., 2006, 16 (10), 2666-2671 relate to EP₁ receptor antagonist compounds.

It is now suggested that a novel group of indole and indazole derivatives are indicated to be useful in treating conditions mediated by the action of PGE₂ at EP₁ receptors. Such conditions include pain, or inflammatory, immunological, bone, neurodegenerative or renal disorders.

Accordingly the present invention provides one or more chemical entities selected from compounds of formula (I):

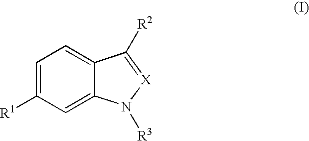

wherein
R¹ represents —CF₃, chlorine or bromine;
R² represents isopropyl, isobutyl or —(CH₂)₂-t-butyl;
X represents CH or N;
R³ represents a group of formula (i)-(iv):

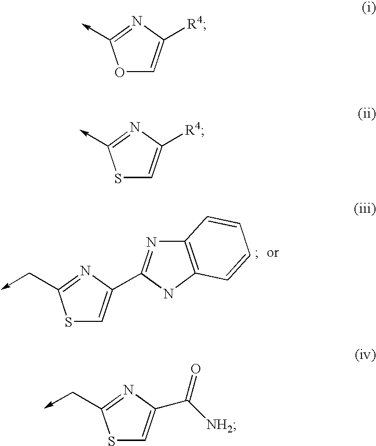

R⁴ represents —CO—NH—R⁵, —NH—CO—R⁶, —CO-pyrrolidinyl or a group of formula (v)-(viii):

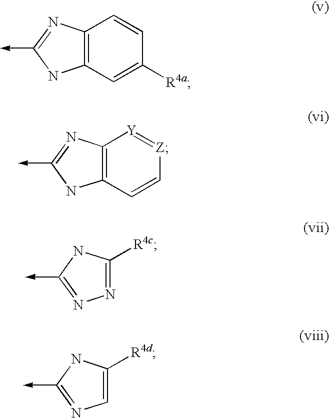

R^{4a} represents hydrogen, —CH₂OH or —CH₂—NR^{a}R^{b};
R^{4c} represents hydrogen or methyl;
R^{4d} represents hydrogen, —CH₂OH or optionally substituted phenyl;
R⁵ represents hydrogen, —(CH₂)₃—OH, pyridyl or optionally substituted phenyl;
R⁶ represents t-butyl, cyclopentyl, NR^{a}R^{b}, pyridyl or optionally substituted phenyl or benzyl;
R^{a} and R^{b} independently represent hydrogen or C₁₋₃ alkyl or R^{a} and R^{b} together with the nitrogen atom which they are attached form an N-pyrrolidinyl, N-piperidinyl or N-morpholinyl ring;
one of Y and Z represents CH and the other represents N;
such that when R² represents —(CH₂)₂-t-butyl, R³ represents a group of formula (iii) or (iv) and such that when R² represent isopropyl, R³ represents a group of formula (ii), R⁴ represents —CO—NH—R⁵ and R⁵ represents 2-aminophenyl; or derivatives thereof.

Optional substituents for phenyl in R^{4d} and R⁶ are selected from optionally substituted C₁₋₆alkyl (e.g. methyl), C₁₋₆alkoxy (e.g. methoxy), cyano, amino, optionally substituted C₁₁-alkylamino, hydroxy, HOC₁₋₄alkyl (e.g. HOCH₂), —CH₂—O—CO—CH₃ and halogen (e.g. fluorine).

Optional substituents for phenyl in R⁵ are selected from optionally substituted C₁₋₆alkyl (e.g. methyl), cyano, amino, optionally substituted C₁₋₆alkylamino, hydroxy, HOC₁₋₄alkyl (e.g. HOCH₂), —CH₂—O—CO—CH₃ and halogen (e.g. fluorine).

Suitably, R¹ represents bromine or chlorine. In a further embodiment, R¹ represents chlorine.

Suitably, R² represents isobutyl.

Suitably, R³ represents a group of formula (ii):

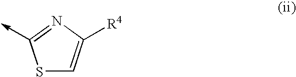

Compounds of formula (I) include the compounds of Examples 1 to 56 and derivatives thereof.

Particular compounds of formula (I) include the compounds of Examples 36, 38, 41, 42, 43 and 45.

A compound of the examples is Example 19.

Certain compounds of the Examples are selective for EP₁ over EP₃. Certain compounds of the Examples have greater than 30 fold selectivity.

Derivatives of the compound of formula (I) include salts, solvates (including hydrates), solvates (including hydrates) of salts, esters and polymorphs of the compound of formula (I). Derivatives of the compounds of formula (I) include pharmaceutically acceptable derivatives.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The present invention also includes isotopically-labelled compounds, which are identical to the compounds of formula (I), except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ²H, ³H, ¹¹C, ¹⁴C, ¹⁸F, ³⁵S, ¹²³I and ¹²⁵I.

Compounds of the present invention and pharmaceutically acceptable derivatives (e.g. salts) of said compounds that contain the aforementioned isotopes and/or other isotopes of other atoms are within the scope of the present invention. Isotopically-labelled compounds of the present invention, for example those into which radioactive isotopes such as ³H and/or ¹⁴C are incorporated, are useful in drug and/or substrate tissue distribution assays. ³H and ¹⁴C are considered useful due to their ease of preparation and detectability. ¹¹C and ¹⁸F isotopes are considered useful in PET (positron emission tomography), and ¹²⁵I isotopes are considered useful in SPECT (single photon emission computerized tomography), all useful in brain imaging. Substitution with heavier isotopes such as ²H can afford certain therapeutic advantages resulting from greater metabolic stability, for example increased in vivo half-life or reduced dosage requirements and, hence, are considered useful in some circumstances. Isotopically labelled compounds of formula (I) of this invention can generally be prepared by carrying out the procedures disclosed in the Schemes and/or in the Examples below, by substituting a readily available isotopically labelled reagent for a non-isotopically labelled reagent.

The following definitions are used herein unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I). In one aspect the term “pharmaceutically acceptable derivative” means any pharmaceutically acceptable salt, solvate or solvate of salt. In an alternative aspect the term “pharmaceutically acceptable derivative” means any pharmaceutically acceptable salt.

It will be appreciated that, for pharmaceutical use, the derivatives referred to above will be pharmaceutically acceptable derivatives, but other derivatives may find use, for example in the preparation of compounds of formula (I) and the pharmaceutically acceptable derivatives thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine; N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

Compounds of formula (I) can be prepared as set forth in the following schemes and in the examples. The following processes form another aspect of the present invention.

Compounds of formula (I) wherein X represents CH, R² represents isobutyl or —(CH₂)₂-t-butyl, R³ represents a group of formula (ii) and R⁴ represents —CO—NH—R⁵ may be prepared by the general route shown in Scheme 1 below:

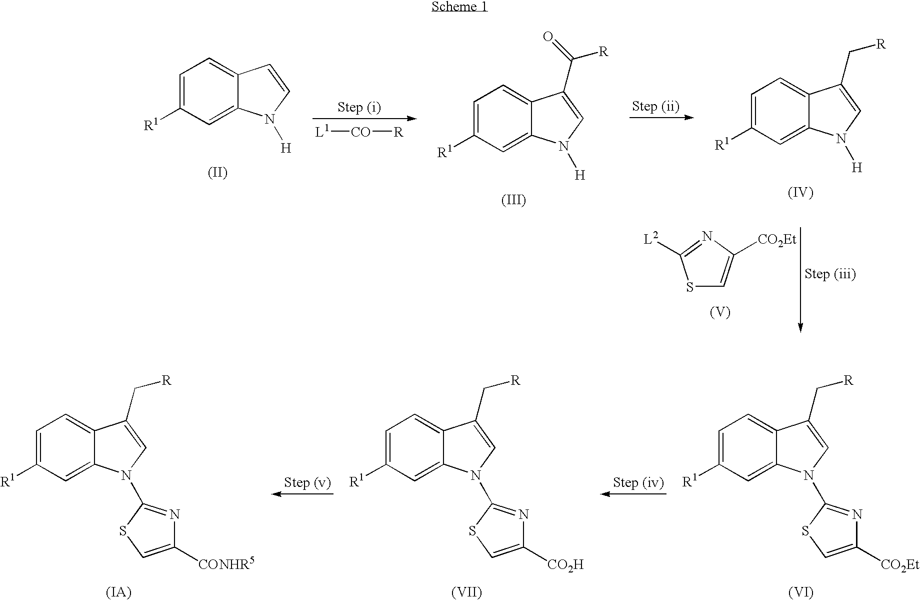

wherein R¹ is as defined above, R represents isopropyl or —CH₂-t-butyl, L¹ represents a suitable leaving group such as a halogen atom (e.g. chlorine) and L² represents a suitable leaving group such as a halogen atom (e.g. bromine).

Step (i) typically comprises reaction of a compound of formula (II) with a compound of formula L¹-CO—R in the presence of suitable reagents, such as methyl magnesium bromide and zinc chloride.

Step (ii) typically comprises a reduction reaction in the presence of a suitable reducing agent, e.g. lithium aluminium hydride or sodium borohydride.

Step (iii) typically comprises a Buchwald coupling reaction between a compound of formula (IV) and a compound of formula (V) in the presence of a suitable catalyst e.g. copper (I) iodide and a suitable base e.g. potassium phosphate and a suitable amine, in the presence of a suitable solvent e.g. toluene.

Step (iv) typically comprises treatment of a compound of formula (VI) with sodium hydroxide.

Step (v) typically comprises treating a compound of formula (VII) with oxalyl chloride followed by a compound of formula NH₂—R⁵. Alternatively, step (v) may also be performed in the presence of EDAC, HOBt and a compound of formula NH₂—R⁵.

Compounds of formula (I) wherein X represents CH, R² represents isopropyl, R³ represents a group of formula (ii) and R⁴ represents —CO—NH—R⁵ may be prepared by the general route shown in Scheme 2 below:

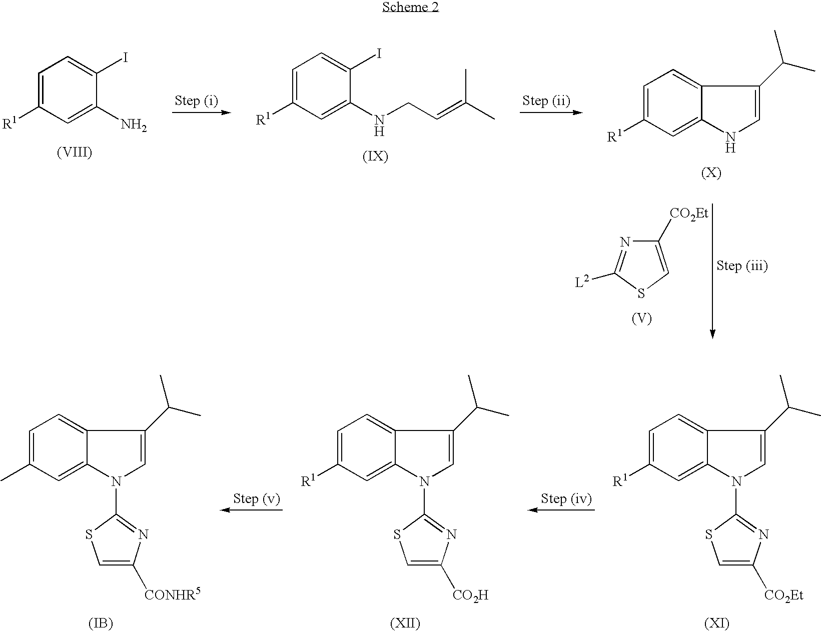

wherein R¹, R⁵ and L² are as defined above.

Step (i) typically comprises reaction of a compound of formula (VIII) with a suitable alkylating reagent, such as 1-bromo-3-methyl but-2-ene, in the presence of a suitable base e.g. lithium diisopropylamide.

Step (ii) typically comprises an intramolecular Heck coupling reaction in the presence of a suitable catalyst e.g. palladium acetate in a suitable solvent e.g. dimethylformamide.

Steps (iii), (iv) and (v) may be performed in an analogous manner to steps (iii), (iv) and (v) in Scheme 1.

It will be appreciated that compounds of formula (I) wherein R⁴ represents —CO-pyrrolidinyl may be prepared in an analogous manner to the procedure described in Schemes 1 and 2 for compounds wherein R⁴ represents —CO—NH—R⁵.

Compounds of formula (I) wherein X represents CH, R² represents isobutyl or —(CH₂)₂-t-butyl, R³ represents a group of formula (ii) and R⁴ represents —NH—CO—R⁶ may be prepared by the general route shown in Scheme 3 below:

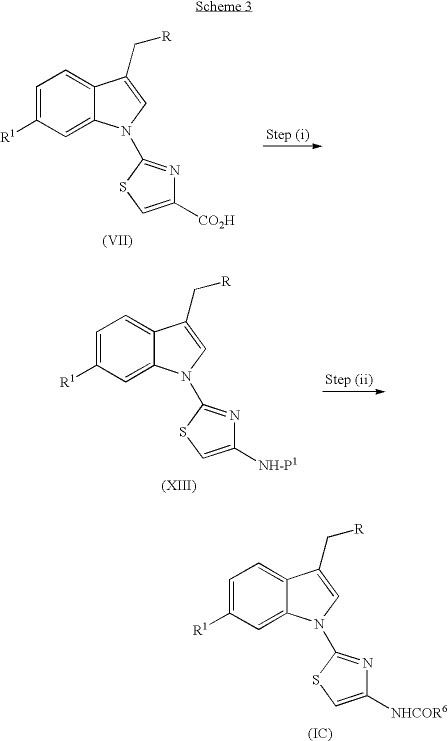

wherein R¹, R and L¹ are as defined above and P¹ represents a suitable protecting group, such as Boc.

Step (i) typically comprises treating a compound of formula (VII) with diphenylphosphoryl azide in the presence of a suitable base, such as triethylamine and a suitable solvent, such as t-butyl alcohol.

Step (ii) typically comprises removal of the protecting group P¹ e.g. if P¹ is Boc then a suitable acid e.g. hydrochloric acid is used to remove protecting group P¹, followed by reaction with a compound of formula L³-CO—R⁶, wherein L³ represents a suitable leaving group such as a halogen atom (e.g. chlorine) and R⁶ is as defined above.

Compounds of formula (I) wherein X represents CH, R² represents isobutyl or —(CH₂)₂-t-butyl, R³ represents a group of formula (ii) and R⁴ represents a group of formula (v) may be prepared by the general route shown in Scheme 4 below:

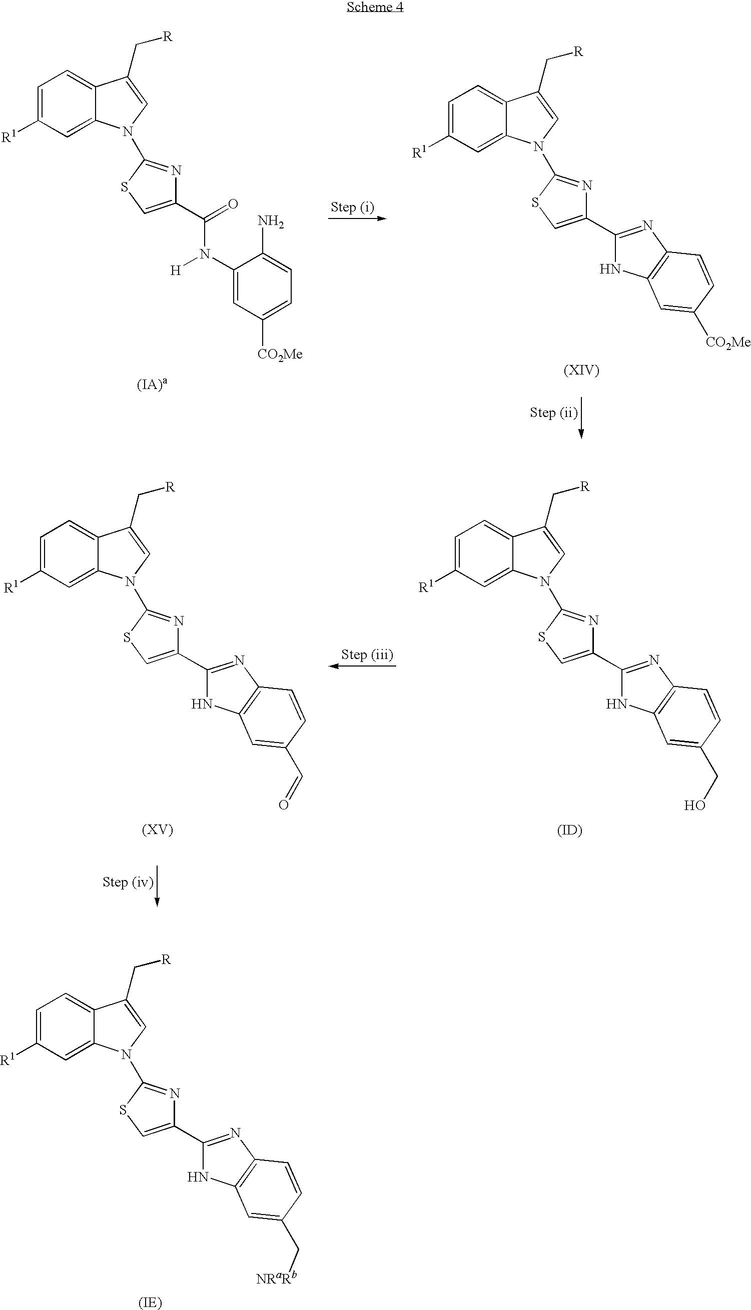

wherein R¹, R and L′ are as defined above.

Step (i) typically comprises treating a compound of formula (IA)^{a} with a mixture of acetic and propionic acid.

Step (ii) typically comprises a reduction reaction in the presence of a suitable reducing agent, e.g. lithium aluminium hydride.

Step (iii) typically comprises an oxidation reaction using a suitable oxidant, for example Dess Martin Periodinane.

Step (iv) typically comprises reaction of a compound of formula (XV) with a compound of formula NHR^{a}R^{b} wherein R^{a} and R^{b} are as defined above, in the presence of a suitable reducing agent e.g. sodium triacetoxyborohydride and a suitable acid e.g. acetic acid in a suitable solvent e.g. dichloromethane.

It will be appreciated that compounds of formula (I) wherein R⁴ represents a group of formula (vi) may be prepared in an analogous manner to the procedure described in Scheme 4 for compounds wherein R⁴ represents a group of formula (v).

Compounds of formula (I) wherein X represents CH, R² represents isobutyl or —(CH₂)₂-t-butyl, R³ represents a group of formula (ii), R⁴ represents a group of formula (viii) and R^{4d} represents —CH₂OH may be prepared by the general route shown in Scheme 5 below:

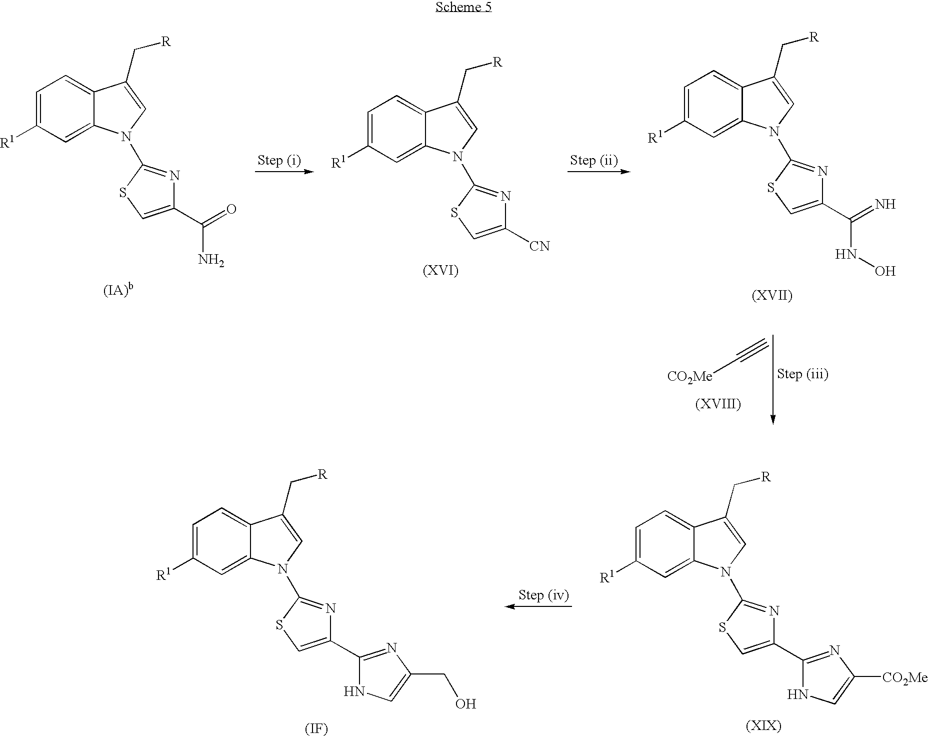

wherein R¹ and R are as defined above.

Step (i) typically comprises treating a compound of formula (IA)_{b} with a suitable dehydrating reagent e.g. phosphoryl chloride.

Step (ii) typically comprises treating a compound of formula (XVI) with hydroxylamine hydrochloride in the presence of a base e.g. sodium bicarbonate, in a suitable solvent e.g. methanol.

Step (iii) typically comprises reacting a compound of formula (XVII) with a compound of formula (XVIII) in the presence of a suitable solvent e.g. methanol, at an elevated temperature.

Step (iv) typically comprises a reduction reaction in the presence of a suitable reducing agent, e.g. lithium aluminium hydride.

Compounds of formula (I) wherein X represents CH, R² represents isobutyl or —(CH₂)₂-t-butyl, R³ represents a group of formula (ii) and R⁴ represents a group of formula (vii) may be prepared by the general route shown in Scheme 6 below:

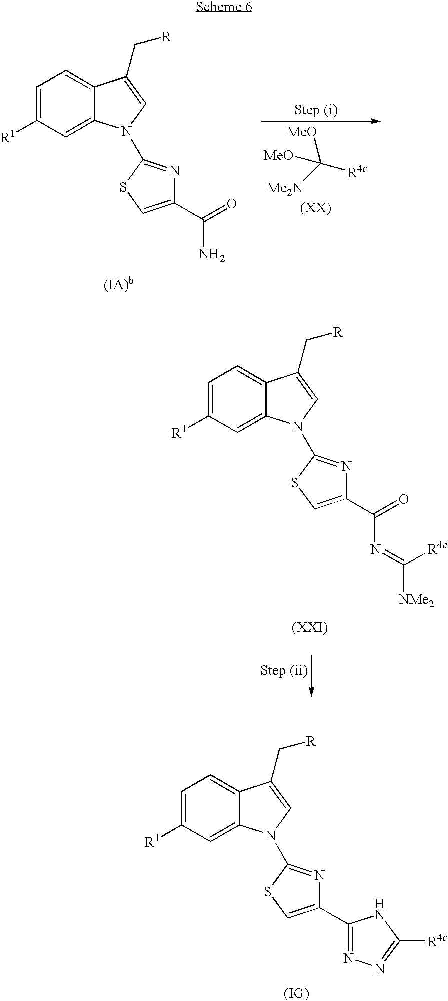

wherein R¹, R and R^{4c} are as defined above.

Step (i) typically comprises reacting a compound of formula (IA)_{b} with a compound of formula (XX) at an elevated temperature.

Step (ii) typically comprises reacting a compound of formula (XXI) with hydrazine hydrate in a suitable solvent e.g. acetic acid at an elevated temperature.

It will be appreciated that compounds of formula (I) wherein R³ represents a group of formula (I) may be prepared in an analogous manner to the procedures described in Schemes 1-6 for compounds wherein R³ represents a group of formula (ii).

Compounds of formula (I) wherein X represents CH, R² represents isobutyl or —(CH₂)₂-t-butyl and R³ represents a group of formula (Iv) may be prepared by the general route shown in Scheme 7 below:

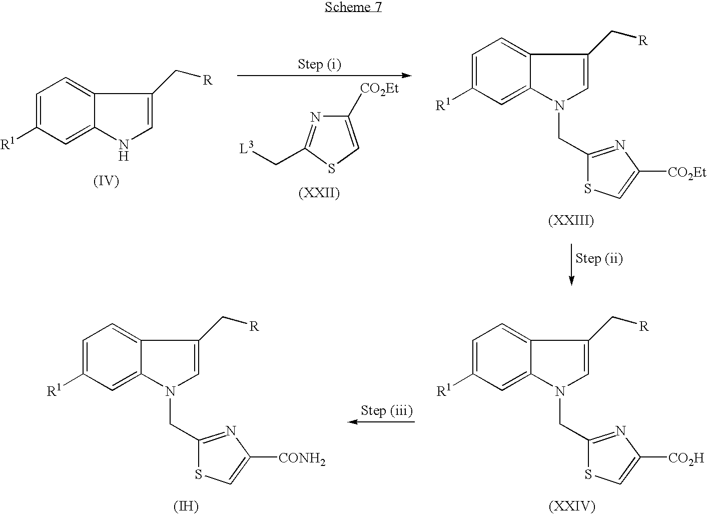

wherein R¹ and R are as defined above and L³ represents a suitable leaving group, such as a halogen atom (e.g. bromine).

Step (i) typically comprises reacting a compound of formula (IV) with a compound of formula (XXII) in the presence of a suitable base e.g. sodium hydride, in a suitable solvent e.g. dimethylformamide.

Step (ii) typically comprises treatment of a compound of formula (XXIII) with aqueous sodium hydroxide in an alcoholic solvent such as methanol or ethanol.

Step (iii) typically comprises activation of the carboxylic acid with oxalyl chloride followed by reaction with ammonia in a suitable solvent e.g. dichloromethane.

Compounds of formula (I) wherein X represents CH, R² represents isobutyl or —(CH₂)₂-t-butyl and R³ represents a group of formula (iii) may be prepared by the general route shown in Scheme 8 below:

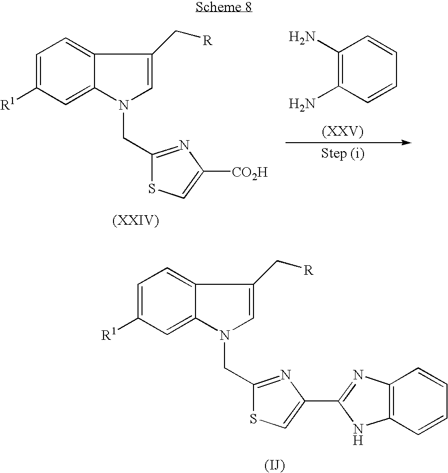

wherein R¹ and R are as defined above.

Step (i) typically comprises reaction of a compound of formula (XXIV) with a compound of formula (XXV) in the presence of EDAC and HOBt in a suitable solvent e.g. dichloromethane to give the intermediate amide which can be dehydrated, for example by heating in acetic acid, to give compounds of formula (IJ).

Compounds of formula (I) wherein X represents N, R² represents isobutyl or —(CH₂)₂-t-butyl, R³ represents a group of formula (ii) and R⁴ represents —CO—NH—R⁵ may be prepared by the general route shown in Scheme 9 below:

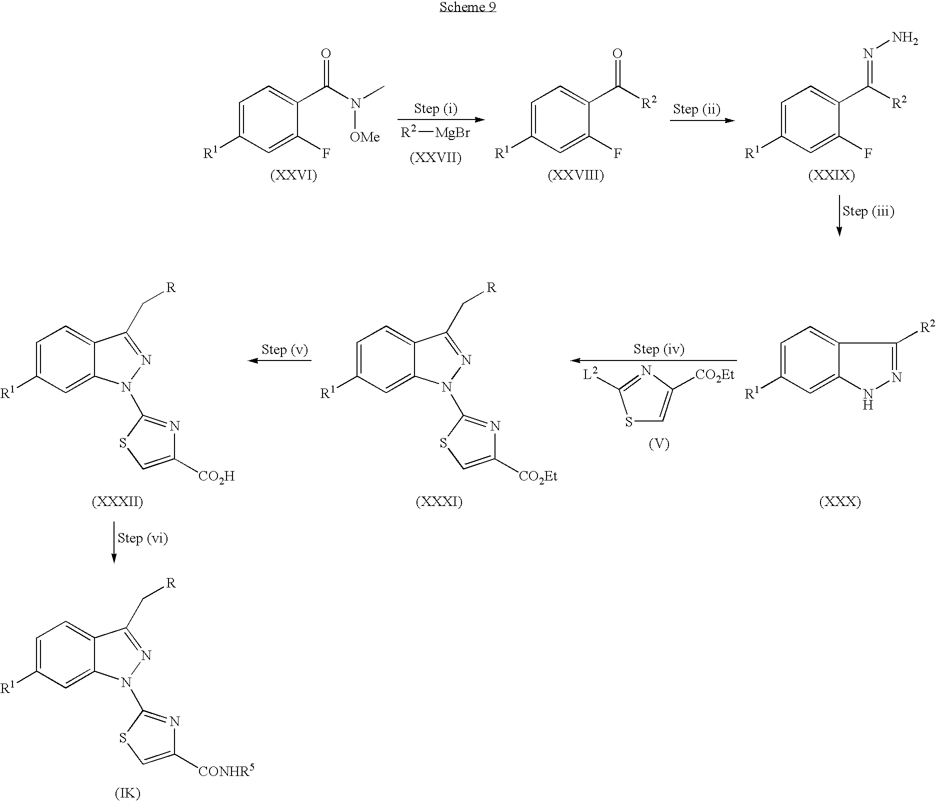

Step (i) comprises reaction of a compound of formula (XXVI) with a compound of formula (XXVII) in a suitable solvent e.g. tetrahydrofuran.

Step (ii) comprises reaction of a compound of formula (XXVIII) with hydrazine hydrate in a suitable solvent e.g. ethanol.

Step (iii) comprises intramolecular cyclisation in a suitable solvent e.g. ethylene glycol at an elevated temperature.

Steps (iv), (v) and (vi) may be performed in an analogous manner to steps (iii), (iv) and (v) in Schemes 1 and 2.

Certain substituents in any of the reaction intermediates and compounds of formula (I) may be converted to other substituents by conventional methods known to those skilled in the art. Examples of such transformations include the hydrolysis of esters and esterification of carboxylic acids. Such transformations are well known to those skilled in the art and are described in for example, Richard Larock, Comprehensive Organic Transformations, 2nd edition, Wiley-VCH, ISBN 0-471-19031-4.

It will be appreciated by those skilled in the art that it may be necessary to protect certain reactive substituents during some of the above procedures. The skilled person will recognise when a protecting group is required. Standard protection and deprotection techniques, such as those described in Greene T.W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

Compounds of formula (II), (V), (VIII), (XVIII), (XX), (XXII), (XXV), (XXVI) and (XXVII) are either commercially available, or may be prepared by known methods.

The compounds of the invention bind to the EP₁ receptor and are antagonists of this receptor. They are therefore considered useful in treating conditions mediated by the action of PGE₂ at EP₁ receptors.

One condition mediated by the action of PGE₂ at EP₁ receptors is pain, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, headache, toothache and dysmenorrhea.

Chronic articular pain conditions include rheumatoid arthritis, osteoarthritis, rheumatoid spondylitis, gouty arthritis and juvenile arthritis.

Pain associated with functional bowel disorders includes non-ulcer dyspepsia, non-cardiac chest pain and irritable bowel syndrome.

Neuropathic pain syndromes include: diabetic neuropathy, sciatica, non-specific lower back pain, multiple sclerosis pain, fibromyalgia, HIV-related neuropathy, post-herpetic neuralgia, trigeminal neuralgia, and pain resulting from physical trauma, amputation, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Other conditions mediated by the action of PGE₂ at EP₁ receptors include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) and cyclooxygenase-2 (COX-2) inhibitors, cardiovascular diseases; neurodegenerative diseases and neurodegeneration, neurodegeneration following trauma, tinnitus, dependence on a dependence-inducing agent such as opoids (e.g. morphine), CNS depressants (e.g. ethanol), psychostimulants (e.g. cocaine) and nicotine; complications of Type I diabetes, kidney dysfunction, liver dysfunction (e.g. hepatitis, cirrhosis), gastrointestinal dysfunction (e.g. diarrhoea), colon cancer, overactive bladder and urge incontinence.

Inflammatory conditions include skin conditions (e.g. sunburn, burns, eczema, dermatitis, psoriasis), ophthalmic diseases such as glaucoma, retinitis, retinopathies, uveitis and of acute injury to the eye tissue (e.g. conjunctivitis), inflammatory lung disorders (e.g. asthma, bronchitis, emphysema, allergic rhinitis, respiratory distress syndrome, pigeon fancier's disease, farmer's lung, chronic obstructive pulmonary disease (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation and other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

Immunological diseases include autoimmune diseases, immunological deficiency diseases or organ transplantation. The compounds of formula (I) are also effective in increasing the latency of HIV infection

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

Cardiovascular diseases include hypertension or myocardiac ischemia; functional or organic venous insufficiency; varicose therapy; haemorrhoids; and shock states associated with a marked drop in arterial pressure (e.g. septic shock).

Neurodegenerative diseases include dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

Complications of Type 1 diabetes include diabetic microangiopathy, diabetic retinopathy, diabetic nephropathy, macular degeneration, glaucoma, nephrotic syndrome, aplastic anaemia, uveitis, Kawasaki disease and sarcoidosis.

Kidney dysfunction includes nephritis, particularly mesangial proliferative glomerulonephritis and nephritic syndrome.

The compounds of formula (I) are also considered useful for the preparation of a drug with diuretic action.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof.

A proposed daily dosage of compounds of formula (I) or their pharmaceutically acceptable derivatives for the treatment of man is from 0.01 to 80 mg/kg body weight, more particularly 0.01 to 30 mg/kg body weight per day, for example 0.1 to 10 mg/kg body weight per day, which may be administered as a single or divided dose, for example one to four times per day. The dose range for adult human beings is generally from 8 to 4000 mg/day, more particularly from 8 to 2000 mg/day, such as from 20 to 1000 mg/day, for example 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

The compounds of formula (I) and their pharmaceutically acceptable derivatives may be formulated for administration in any suitable manner. They may be formulated for administration by inhalation or for oral, topical, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 (cyclooxygenase-2) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO99/012930); 5-lipoxygenase inhibitors; NSAIDs (non-steroidal anti-inflammatory drugs) such as diclofenac, indomethacin, nabumetone or ibuprofen; leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists; ligands for the α₂δ-subunit of voltage gated calcium channels, such as gabapentin and pregabalin; tricyclic antidepressants such as amitriptyline; neurone stabilising antiepileptic drugs; mono-aminergic uptake inhibitors such as venlafaxine; opioid analgesics; local anaesthetics; 5HT, agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabanoid receptor ligands; bradykinin receptor ligands; vanilloid receptor ligand; and purinergic receptor ligands, including antagonists at P2X₃, P2X_{2/3}, P2X₄, P2X₇ or P2X_{4/7}. When the compounds are used in combination with other therapeutic agents, the compounds may be administered either sequentially or simultaneously by any convenient route.

Additional COX-2 inhibitors are disclosed in U.S. Pat. Nos. 5,474,995 5,633,272; 5,466,823, 6,310,099 and 6,291,523; and in WO 96/25405, WO 97/38986, WO 98/03484, WO 97/14691, WO99/12930, WO0/26216, WO00/52008, WO00/38311, WO01/58881 and WO02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art. No toxicological effects have currently been observed with the compounds of the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

### Abbreviations

Solid phase extraction (SPE); liquid chromatography/mass spectrometry (LCMS, LC/MS & LC-MS); MDAP (Mass Directed Auto Preparation); NMR (nuclear magnetic resonance); s, d, t, dd, m, b (singlet, doublet, triplet, doublet of doublets, multiplet, broad); Ph, Me, Et, Pr, Bu, Bn (phenyl, methyl, ethyl, propyl, butyl, benzyl), tetrahydrofuran (THF), dichloromethane (DCM), N,N-dimethylformamide (DMF), h (hours), ethylenediaminetetraacetic acid (EDTA), 1-(3-dimethylaminopropyl)-1-ethylcarbodiimide hydrochloride (EDC & EDAC), 1-hydroxybenzotriazole (HOBt & HOBT), ultraviolet (UV), room temperature (RT), retention time (Rt), minutes (min), EtOAc (ethyl acetate), Et₂O (diethyl ether), MeCN (acetonitrile).

### Purification of Reaction Products

Conventional techniques may be used herein for work up of reactions and purification of the products of the Examples.

References in the Examples below relating to the drying of organic layers or phases may refer to drying the solution over magnesium sulfate or sodium sulfate and filtering off the drying agent in accordance with conventional techniques. Products may generally be obtained by removing the solvent by evaporation under reduced pressure.

Purification of the Examples may be carried out by conventional methods such as chromatography and/or recrystallisation using suitable solvents. Chromatographic methods are known to the skilled person and include e.g. column chromatography, flash chromatography, HPLC (high performance liquid chromatography), and MDAP (mass directed autopreparation, also referred to as mass directed LCMS purification). MDAP is described in e.g. W. Goetzinger et al, Int. J. Mass Spectrom., 2004, 238, 153-162.

The terms “Biotage®” and “Flashmaster II®” when used herein refer to commercially available automated purification systems using pre-packed silica gel cartridges.

### LCMS

The following LCMS conditions were used during the preparation of the examples.

### Software

Waters MassLynx version 4.0 SP2

### Column

The column used is a Waters Atlantis, the dimensions of which are 4.6 mm×50 mm. The stationary phase particle size is 3 m.

### Solvents

A: Aqueous solvent=Water+0.05% Formic Acid
B: Organic solvent=Acetonitrile+0.05% Formic Acid

### Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

All retention times are measured in minutes.

### Description 1

### 1-(6-Chloro-1H-indol-3-yl)-2-methyl-1-propanone (D1)

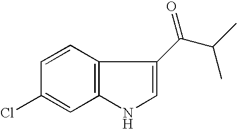

3M Methylmagnesium bromide in ether (1.7 ml, 5.1 mmol) were added to a stirred solution of 6-chloroindole (758 mg, 5 mmol) in dry ether (10 ml) under argon producing a two phase mixture and gas evolution. After stirring for 15 minutes 1M zinc chloride in ether (5 ml) was added and the mixture stirred for 30 minutes before isobutyryl chloride (533 mg, 5 mmol) was added rapidly with vigorous stirring. The mixture was stirred for 30 minutes and quenched by addition of saturated ammonium chloride solution then diluted with ethyl acetate. The organic phase was dried (magnesium sulphate), evaporated, triturated with ether and filtered to give the title compound as a pink solid (710 mg).

LCMS: Rt=2.89 min, [MH⁺] 222.18, 224.17

### Descriptions 2-4 (D2-D4)

The following compounds were prepared by an analogous method to that described for D1 using indole or a 6-substituted indole and the appropriate chloride:

| Name | Structure | Data |
|---|---|---|
| 1-(6-Chloro-1H-indol-3-yl)-3,3- dimethyl-1-butanone (D2) | 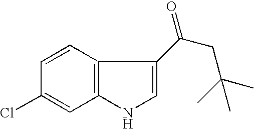 | LCMS Rt = 3.26 min [MH]⁺ 250.27 252.26 |
| 1-(6-Bromo-1H-indol-3-yl)-2- methyl-1-propanone (D3) | 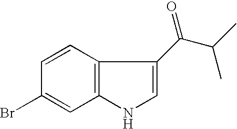 | |
| 2-Methyl-1-[6-(trifluoromethyl)- 1H-indol-3-yl]-1-propanone (D4) | 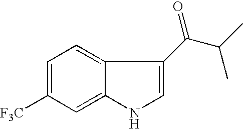 | |

### Description 5

### 6-Chloro-3-isobutyl-1H-indole (D5)

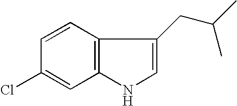

1M Lithium aluminium hydride in THF (7 ml, 7 mmol) was added to a stirred solution of 1-(6-chloro-1H-indol-3-yl)-2-methyl-1-propanone (705 mg, 3.21 mmol; may be prepared as described in D1) in THF (15 ml) and heated at 55° C. for four hours. The solution was cooled and quenched by careful addition of 2M sodium hydroxide and ether. The organic phase was dried (magnesium sulphate), evaporated and purified on a Biotage column eluting with (1:9) ethyl acetate/hexane to give the title compound as a colourless oil which crystallised on scratching (663 mg).

LCMS: Rt=3.66 min, [MH⁺] 208.22, 210.24

### Description 6

### 6-Chloro-3-(3,3-dimethylbutyl)-1H-indole (D6)

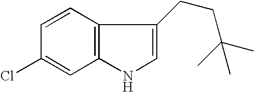

D6 was prepared by reduction of the appropriate ketone using an analogous procedure to that described for D5.

LCMS Rt=3.98 min, [MH]⁺ 236.26, 238.26

### Description 7

### 3-Isobutyl-6-(trifluoromethyl)-1H-indole (D7)

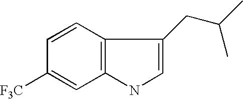

2-Methyl-1-[6-(trifluoromethyl)-1H-indol-3-yl]-1-propanone (2.3 g, 9 mmol; may be prepared as described in D4) was dissolved in anhydrous THF (30 ml), cooled to 0° C. and treated with 1M borane in THF (21 ml, 21 mmol) dropwise at 0° C. The mixture was stirred at room temperature for 1 hr, cooled in ice, treated with 1.5M hydrochloric acid (15 ml) dropwise, extracted with ethyl acetate, dried over sodium sulphate and evaporated to give the title compound (1.52 g) as a green solid.

### Description 8

### 6-Bromo-3-isobutyl-1H-indole (D8)

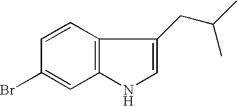

D8 was prepared in an analogous manner to that described in D7.

### Description 9

### (5-Chloro-2-iodophenyl)(3-methyl-2-buten-1-yl)amine (D9)

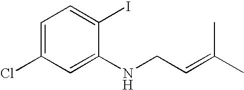

2M Lithium diisopropylamide in heptane/THF/ethylbenzene (4.16 ml, 8.32 mmol) were added to a stirred solution of 5-chloro-2-iodoaniline in dry THF (35 ml) at −78° C. under argon then allowed to warm to 0° C. before being cooled to −78° C. and 1-bromo-3-methylbut-2-ene (1.364 g, 9.15 mmol) was added. The resulting solution was stirred for ten minutes at −78° C. then allowed to warm to room temperature and stirred for one hour before quenching with water/ether. The organic phase was dried (magnesium sulphate), evaporated and purified on a Biotage column eluting with hexane to give the title compound as a pale coloured oil (2.1 g).

LCMS: Rt=4.16 min.

### Description 10

### 6-Chloro-3-isopropyl-1H-indole (D10)

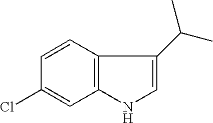

A mixture of (5-chloro-2-iodophenyl)(3-methyl-2-buten-1-yl)amine (2.15 g, 6.7 mmol; may be prepared as described in D9), palladium acetate (30 mg, 0.134 mmol), tetrabutylammonium bromide (2.157 g, 6.7 mmol) and triethylamine (1.69 g, 16.73 mmol) in dimethylformamide (12 ml) was stirred and heated at 80° C. under argon for one hour. The resulting mixture was cooled, diluted with ether/water and the organic phase was dried (magnesium sulphate), evaporated and purified on a Biotage column eluting with ethyl acetate/hexane (1:99) then recrystallised from hexane to give the title compound as a white solid (710 mg).

LCMS: Rt=3.52 min, [MH⁺] 194.21, 196.18.

### Description 11

### 1-(4-Chloro-2-fluorophenyl)-3-methyl-1-butanone (D11)

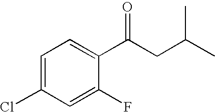

4-Chloro-2-fluoro-N-methyl-N-(methyloxy)benzamide (6.1 g, 28.05 mmol) in THF (20 ml) was added over 10 minutes to isobutylmagnesium bromide (prepared from 800 mg, 32.92 mmol of magnesium and 4.11 g, 30 mmol of isobutyl bromide in 50 ml of THF). Stirred for 30 minutes at room temperature then heated at 60° C. for 4 hours. The solution was cooled, diluted with 2M hydrochloric acid/ether and the organic phase dried (magnesium sulphate), evaporated and purified on a Biotage column eluting with ethyl acetate/hexane (5:195) to give the title compound as colourless oil (806 mg).

LCMS: Rt=3.58 min.

### Description 12

### 1-(4-Chloro-2-fluorophenyl)-3-methyl-1-butanone hydrazone (D12)

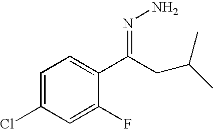

Hydrazine hydrate (400 mg, 8 mmol) was added to a solution of 1-(4-chloro-2-fluorophenyl)-3-methyl-1-butanone (800 mg, 3.73 mmol; may be prepared as described in D11) in ethanol (10 ml) and left at room temperature overnight. The resulting solution was evaporated, dissolved in ethyl acetate/water and the organic phase dried (magnesium sulphate), evaporated and purified on a Biotage column eluting with ethyl acetate/hexane (15:85) to give the title compound as colourless oil (585 mg).

LCMS: Rt=2.90, 2.97 min [MH⁺] 229.22, 231.21.

### Description 13

### 6-Chloro-3-isobutyl-1H-indazole (D13)

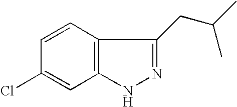

1-(4-Chloro-2-fluorophenyl)-3-methyl-1-butanone hydrazone (580 mg; may be prepared as described in D12) in ethylene glycol (5 ml) was stirred and heated at 165° C. for 3 hours. The solution was cooled diluted with ether/water and the organic phase washed with water, dried (magnesium sulphate), evaporated and purified on a Biotage column eluting with ethyl acetate/hexane (1:7) to give the title compound as white solid (170 mg).

LCMS: Rt=3.10 min [MH⁺] 209.24, 211.23.

### Description 14

### Ethyl 2-[6-chloro-3-isobutyl-1H-indol-1-yl]-1,3-thiazole-4-carboxylate (D14)

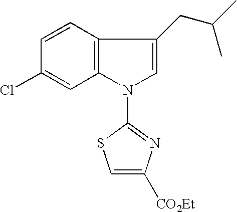

A mixture of 6-chloro-3-isobutyl-1H-indole (660 mg, 3.18 mmol; may be prepared as described in D5), ethyl 2-bromo-4-thiazolecarboxylate (750 mg, 3.18 mmol), potassium phosphate (1.416 g, 6.68 mmol), copper(I) iodide (30 mg, 0.16 mmol) and (1R,2R)-N,N′-dimethyl-1,2-cyclohexanediamine (53 mg, 0.37 mmol) in toluene (4 ml) was stirred and heated at 110° C. under argon for 48 hours when a further quantity of copper(I) iodide (30 mg, 0.16 mmol) and (1R,2R)-N,N′-dimethyl-1,2-cyclohexanediamine (53 mg, 0.37 mmol) was added. After heating for a further 24 hours the mixture was cooled, diluted with ether/water and the organic phase dried (magnesium sulphate), evaporated and purified on a Biotage column eluting with (8:92) ethyl acetate/hexane to give the title compound as a white solid (380 mg).

LCMS: Rt=4.14 min, [MH⁺] 363.24, 363.23.

### Descriptions 15-19 (D15-D19)

The following compounds were prepared by an analogous method to that described in D14 using the appropriate indole or indazole and a bromo heterocycle or a bromobenzene. Ethyl 2-bromo-1,3-oxazole-4-carboxylate was prepared as described in Organic Letters 4(17), 2905-2907 (2002)

| Name | Structure | Data |
|---|---|---|
| Ethyl 2-[6-chloro-3-(3,3- dimethylbutyl)-1H-indol-1-yl]- 1,3-thiazole-4-carboxylate (D15) | 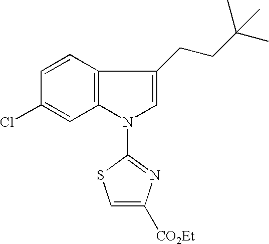 | LCMS Rt = 4.55 min [MH]⁺ 391.24, 393.24 |
| Ethyl 2-[6-bromo-3-isobutyl- 1H-indol-1-yl]-1,3-thiazole-4- carboxylate (D16) | 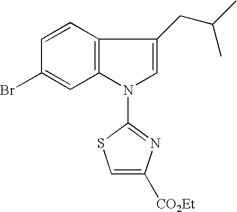 | LCMS Rt = 4.27 min [MH]⁺ 409.14, 410.14 |
| Ethyl 2-[6-chloro-3-isobutyl-1H- indol-1-yl]-1,3-oxazole-4- carboxylate (D17) | 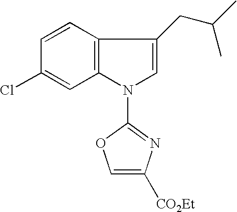 | LCMS Rt = 4.18 min [MH]⁺ 347.1, 349.1 |
| Ethyl 2-[6-chloro-3-isopropyl- 1H-indol-1-yl]-1,3-thiazole-4- carboxylate (D18) | 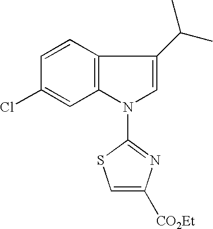 | LCMS Rt = 4.19 min [MH]⁺ 349.16, 351.15 |
| Ethyl 2-[6-chloro-3-isobutyl-1H- indazol-1-yl]-1,3-thiazole-4- carboxylate (D19) | 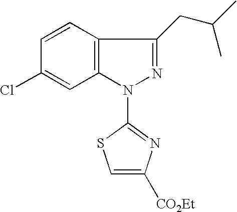 | LCMS Rt = 4.23 min [MH]⁺ 364.17, 366.16 |

### Description 20

### Ethyl 2-[3-isobutyl-6-(trifluoromethyl)-1H-indol-1-yl]-1,3-thiazole-4-carboxylate (D20)

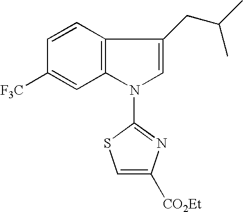

A mixture of 3-isobutyl-6-(trifluoromethyl)-1H-indole (480 mg, 2 mmol; may be prepared as described in D7), ethyl 2-bromo-4-thiazolecarboxylate (472 mg, 2 mmol), potassium phosphate (850 mg, 4 mmol), copper(I) iodide (19 mg, 0.1 mmol) and (1R,2R)-N,N′-dimethyl-1,2-cyclohexanediamine (33 mg, 0.23 mmol) in toluene (10 ml) was stirred and heated at 110° C. over night. A further quantity of copper(I) iodide (19 mg) and (1R,2R)-N,N′-dimethyl-1,2-cyclohexanediamine (33 mg) was added. After heating for a further 5 hours the mixture was washed with sodium bicarbonate solution, and the organic phase dried, evaporated and purified by flash chromatography eluting with 1-19% ethyl acetate/hexane, then triturated with hexane to give the title compound as a white solid (35 mg).

LCMS: Rt=4.19 min [MH]⁺ 397.3.

### Description 21

### Ethyl 2-{[6-chloro-3-isobutyl-1H-indol-1-yl]methyl}-1,3-thiazole-4-carboxylate (D21)

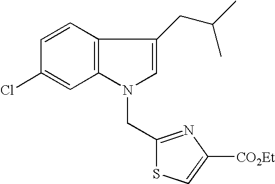

60% Sodium hydride (100 mg, 2.5 mmol) was added to a stirred solution of 6-chloro-3-isobutyl-1H-indole (519 mg, 2.5 mmol; may be prepared as described in D5) in dimethylformamide (8 ml) under argon and stirred for 10 minutes when ethyl 2-(bromomethyl)-1,3-thiazole-4-carboxylate (625 mg, 2.5 mmol) were added. The solution was stirred at room temperature for one hour then diluted with ether/water. The organic phase was washed three times with water, dried (magnesium sulphate), evaporated and purified on a Biotage column eluting with (20:80) ethyl acetate/hexane then triturated with hexane to give the title compound as a white solid (388 mg).

LCMS: Rt=3.83 min, [MH⁺] 377.19, 379.18.

### Description 22

### 2-[6-Chloro-3-(3,3-dimethylbutyl)-1H-indol-1-yl]-1,3-thiazole-4-carboxylic acid (D22)

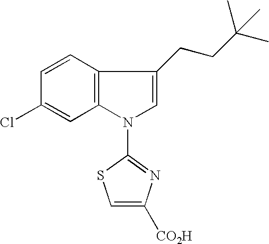

Ethyl 2-[6-chloro-3-(3,3-dimethylbutyl)-1H-indol-1-yl]-1,3-thiazole-4-carboxylate (280 mg, 0.72 mmol; may be prepared as described in D15) was dissolved in ethanol (10 ml) and 2M sodium hydroxide (2 ml) added and left at room temperature for 2 hours. The solution was evaporated to dryness, dissolved in ethyl acetate/2M hydrochloric acid and the organic phase dried (magnesium sulphate), evaporated and triturated with ether/hexane to give the title compound as an off-white solid (158 mg).

LCMS: Rt=4.03 min, [MH⁺] 363.18, 365.23.

### Description 23-28 (D23-D28)

The following compounds were prepared by treating the appropriate ester with sodium hydroxide using an analogous procedure to that described in D22.

| Name | Structure | Data |
|---|---|---|
| 2-[6-Chloro-3-isopropyl- 1H-indol-1-yl]-1,3- thiazole-4-carboxylic acid (D23) | 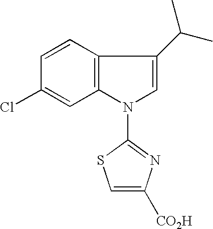 | LCMS Rt = 3.53 min [MH]⁺ 321.15, 323.16 |
| 2-[6-Bromo-3-isobutyl- 1H-indol-1-yl]-1,3- thiazole-4-carboxylic acid (D24) | 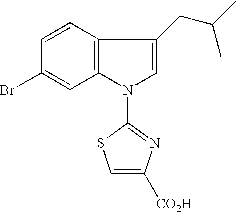 | LCMS Rt = 3.65 min [MH]⁺ 379.10, 382.15 |
| 2-[6-Chloro-3-isobutyl- 1H-indol-1-yl]-1,3- oxazole-4-carboxylic acid (D25) | 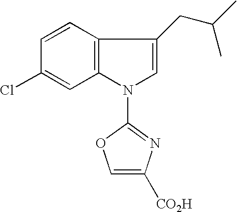 | LCMS Rt = 3.57 min [MH]⁺ 319.20, 321.19 |
| 2-{[6-Chloro-3-isobutyl- 1H-indol-1-yl]methyl}-1,3- thiazole-4-carboxylic acid (D26) | 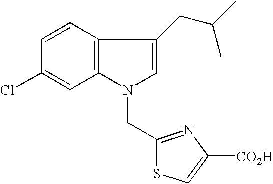 | LCMS Rt = 3.31 min [MH]⁺ 349.23, 351.22 |
| 2-[6-Chloro-3-isobutyl- 1H-indazol-1-yl]-1,3- thiazole-4-carboxylic acid (D27) | 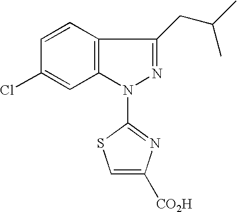 | LCMS Rt = 3.51 min [MH]⁺ 336.20, 338.20 |
| 2-[3-Isobutyl-6- (trifluoromethyl)-1H-indol- 1-yl]-1,3-thiazole-4- carboxylic acid (D28) | 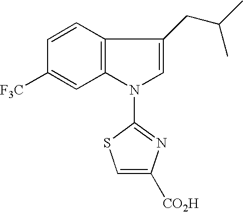 | LCMS Rt = 3.66 min [MH]⁺ 369.1 |

### Description 29

### 2-[6-Chloro-3-isobutyl-1H-indol-1-yl]-1,3-thiazole-4-carboxylic acid (D29)

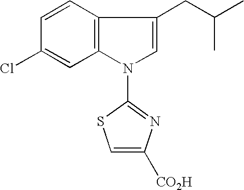

Ethyl 2-[6-chloro-3-isobutyl-1H-indol-1-yl]-1,3-thiazole-4-carboxylate (2.6 g not clean; may be prepared as described in D14) was dissolved in ethanol (20 ml) and 2M sodium hydroxide (10 ml) added and stirred at 50° C. for 1 hr. The solution was cooled, evaporated to dryness, diluted with water and extracted with diethyl ether (×3). The aqueous phase was then acidified with 2M hydrochloric acid, extracted with ethyl acetate (×3), the organic phase was dried (MgSO₄) and evaporated to give the title compound as pale yellow solid (1.5 g).

LCMS: Rt=3.67 min, [MH⁺] 335.1, 337.1, [MH] 333.2, 335.1.

### Description 30

### 1,1-Dimethylethyl {2-[6-chloro-3-isobutyl-1H-indol-1-yl]-1,3-thiazol-4-yl}carbamate (D30)

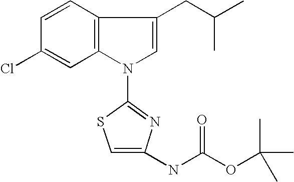

A mixture of 2-[6-chloro-3-isobutyl-1H-indol-1-yl]-1,3-thiazole-4-carboxylic acid (1 g, 2.99 mmol; may be prepared as described in D29), Et₃N (498 μl, 3.59 mmol), diphenyl phosphoryl azide (713 μl, 3.28 mmol) in tert-butanol (25 ml) was refluxed for 4 hr. The solution was cooled, evaporated and purified on the Flash Master II using 8% of ethyl acetate in hexane to give the title compound (1 g).

LCMS: Rt=4.39 min, [MH⁺] 406.2, 408.2, [MH] 404.2, 406.15.

### Description 31

### 2-[6-Chloro-3-isobutyl-1H-indol-1-yl]-1,3-thiazol-4-amine hydrochloride (D31)

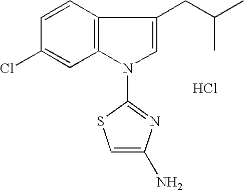

1,1-Dimethylethyl {2-[6-chloro-3-isobutyl-1H-indol-1-yl]-1,3-thiazol-4-yl}carbamate (1 g, 2.46 mmol; may be prepared as described in D30) was dissolved in dioxane and 4M HCl in dioxane (8 ml) was added. The mixture was stirred at room temperature for 2 hr, and then left in the fridge over the weekend. The solvent was evaporated and the residue was triturated with ether to give the title compound as yellow solid (600 mg).

LCMS: Rt=2.83 min, [MH⁺] 306.1, 308.1, 309.1.

### Description 32

### 4-({[2-(6-Chloro-3-isobutyl-1H-indol-1-yl)-1,3-thiazol-4-yl]amino}carbonyl)benzyl acetate (D32) (Example 56)

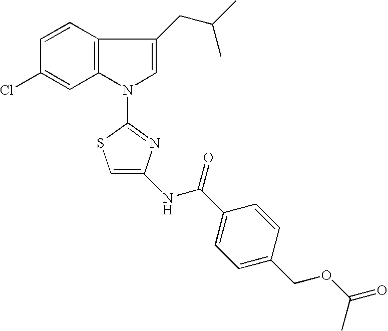

Oxalyl chloride (0.029 ml) was added to a solution of 4-acetoxymethylbenzoic acid (43 mg, 0.22 mmol) in dichloromethane (3 ml) and one drop of dimethylformamide, stirred at room temperature for 30 mins and evaporated to dryness. The residue was dissolved in dichloromethane (3 ml) and added to a solution of 2-[6-chloro-3-isobutyl-1H-indol-1-yl]-1,3-thiazol-4-amine hydrochloride (68 mg, 0.2 mmol; may be prepared as described in D31) in dichloromethane (2 ml) and triethylamine (0.061 ml, 0.44 mmol). The solution was stirred at room temperature for 1 hr, washed with 2M hydrochloric acid, saturated sodium bicarbonate solution, dried, evaporated and purified by flash chromatography on silica gel eluting with ethyl acetate/hexane 1:5 to give the title compound (45 mg) as a yellow solid.

LCMS: Rt=4.33 min [M+H] 482.2.

### Description 33

### Methyl 2-{2-[6-chloro-3-isobutyl-1H-indol-1-yl]-1,3-thiazol-4-yl}-1H-benzimidazole-4-carboxylate (D33)

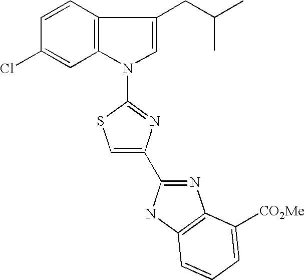

A mixture of 2-[6-chloro-3-isobutyl-1H-indol-1-yl]-1,3-thiazole-4-carboxylic acid (483 mg, 1.31 mmol; may be prepared as described in D29), 1-hydroxybenzotriazole (224 mg, 1.66 mmol), EDAC (318 mg, 1.66 mmol) and methyl 2,3-diaminobenzoate (269 mg, 1.62 mmol) in dichloromethane (8 ml) was stirred at room temperature for 4 hr. The resulting solution was diluted with DCM, washed with saturated sodium bicarbonate and with water then dried, evaporated, triturated with hexane/ether. The resulting off-white solid was Heated at 110° C. in CH₃COOH (5 ml) for 2 hours and the solution cooled, diluted with DCM and washed three times with saturated sodium bicarbonate. The organic phase was dried, evaporated, triturated with diethyl ether to give the title compound as an off-white solid.

LCMS: Rt=4.52 min, [MH⁺] 465.1, 467.1, [MH] 463.2, 465.2.

### Description 34

### {2-[6-Chloro-3-isobutyl-1H-indol-1-yl]-1,3-thiazol-4-yl}methanol (D34)

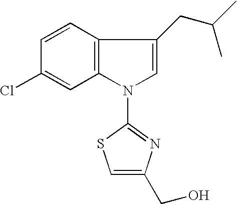

1M Lithium aluminium hydride (1.5 ml) was added to a solution of ethyl 2-[6-chloro-3-isobutyl-1H-indol-1-yl]-1,3-thiazole-4-carboxylate (363 mg, 1 mmol; may be prepared as described in D14) in dry THF (10 ml) under argon and stirred for 30 minutes. 2M sodium hydroxide was added carefully followed by ether and the organic phase was dried (magnesium sulphate), evaporated and purified on a Biotage column eluting with ethyl acetate/hexane (1:4) to give the title compound as a colourless oil (286 mg).

LCMS: Rt=3.83 min, [MH⁺] 321.19, 323.18.

### Description 35

### 2-[6-Chloro-3-isobutyl-1H-indol-1-yl]-1,3-thiazole-4-carbaldehyde (D35)

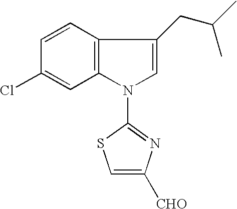

Dess-Martin periodinane (318 mg, 0.75 mmol) was added to a stirred solution of {2-[6-chloro-3-isobutyl-1H-indol-1-yl]-1,3-thiazol-4-yl}methanol (241 mg, 0.75 mmol; may be prepared as described in D34) in dichloromethane (5 ml) under argon and stirred for 30 minutes. The resulting solution was washed with a solution of sodium thiosulphate (1.5 g) in saturated sodium bicarbonate (10 ml) then dried (magnesium sulphate), evaporated and purified on a Biotage column eluting with ethyl acetate/hexane (1:4) to give the title compound as a white solid (191 mg).

LCMS: Rt=4.05 min, [MH⁺] 319.20, 321.19.

### Description 36

### 2-{2-[6-Chloro-3-isobutyl-1H-indol-1-yl]-1,3-thiazol-4-yl}-1H-benzimidazole-5-carbaldehyde (D36)

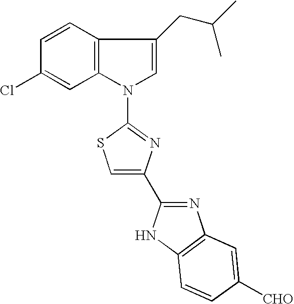

Dess-Martin periodinane (1.24 g, 3 mmol) was added to a stirred solution of (2-{2-[6-chloro-3-isobutyl-1H-indol-1-yl]-1,3-thiazol-4-yl}-1H-benzimidazol-5-yl)methanol (750 mg, 1.72 mmol; may be prepared as described in E45) in dichloromethane (20 ml) under argon and stirred for 30 minutes. The resulting solution was washed with a solution of sodium thiosulphate (1.5 g) in saturated sodium bicarbonate (10 ml) then dried (magnesium sulphate), evaporated and purified on a Biotage column eluting with ethyl acetate/hexane (3:2). The product was triturated with ether to give the title compound as a white solid (715 mg).

LCMS: Rt=4.09 min, [MH⁺] 435.19, 437.19.

### Description 37

### 2-[6-Chloro-3-isobutyl-1H-indol-1-yl]-1,3-thiazole-4-carbonitrile (D37)

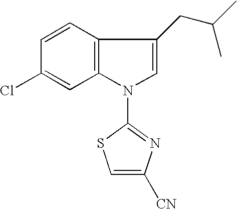

2-[6-Chloro-3-isobutyl-1H-indol-1-yl]-1,3-thiazole-4-carboxamide (915 mg, 2.74 mmol; may be prepared as described in E30) in phosphoryl chloride (5 ml) was stirred and heated at 60° C. for 3 hours. The solution was cooled, poured onto ice diluted with ethyl acetate and the organic phase was washed with saturated sodium bicarbonate, dried (magnesium sulphate), evaporated and triturated with 1:1 ether hexane to give the title compound as a white solid (810 mg).

LCMS: Rt=4.20 min, [MH⁺] 316.17, 318.14.

### Description 38

### Methyl 2-[6-chloro-3-isobutyl-1H-indol-1-yl]-1,3-thiazole-4-carboximidoate hydrochloride (D38)

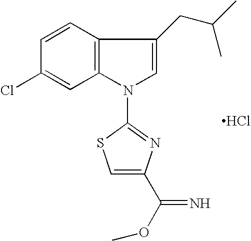

60% Sodium hydride (25 mg, 0.63 mmol) was added to a solution of 2-[6-chloro-3-isobutyl-1H-indol-1-yl]-1,3-thiazole-4-carbonitrile (770 mg, 2.44 mmol; may be prepared as described in D37) in methanol (20 ml) and left over the weekend. The resulting solution was evaporated and the residue dissolved in dichloromethane and acidified with 1M hydrogen chloride in ether. After filtration the filtrate was evaporated to give the title compound as an off-white solid (825 mg).

LCMS: Rt=3.59 min, [MH⁺] 348.23, 350.22.

### Description 39

### 2-[6-Chloro-3-isobutyl-1H-indol-1-yl]-1,3-thiazole-4-carboximidamide (D39)

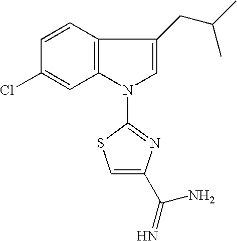

Methyl 2-[6-chloro-3-isobutyl-1H-indol-1-yl]-1,3-thiazole-4-carboximidoate hydrochloride (760 mg, 1.98 mmol; may be prepared as described in D38) was dissolved in THF/7M methanolic ammonia (1:1, 50 ml) and then a further 60 ml of 7M methanolic ammonia was added. The mixture was stirred at room temperature for 7 days then evaporated to dryness. The residue was treated with dichloromethane and 1M sodium hydroxide and the solid filtered off. The dichloromethane solution was dried with magnesium sulphate and evaporated to give a white solid which was triturated with dichloromethane, filtered off and combined with the solid isolated above to give the title compound (410 mg).

LCMS: Rt=2.34 min, [MH⁺] 333.22, 335.21.

### Description 40

### 2-[6-Chloro-3-isobutyl-1H-indol-1-yl]-N-hydroxy-1,3-thiazole-4-carboximidamide (D40)

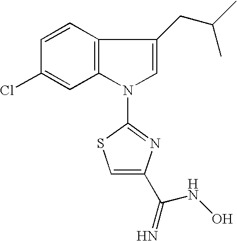

A mixture of 2-[6-chloro-3-isobutyl-1H-indol-1-yl]-1,3-thiazole-4-carbonitrile (890 mg, 2.82 mmol; may be prepared as described in D37), sodium bicarbonate (286 mg, 3.4 mmol) and hydroxylamine hydrochloride (236 mg, 3.4 mmol) in methanol (10 ml) was stirred and refluxed for 4 hours. The mixture was cooled, evaporated, dissolved in dichloromethane/water and the organic phase was dried (magnesium sulphate), evaporated and triturated with ether to give the title compound as a white solid (980 mg).

LCMS: Rt=3.46 min, [MH⁺] 349.1, 351.1.

### Description 41

### Methyl 2-{2-[6-chloro-3-isobutyl-1H-indol-1-yl]-1,3-thiazol-4-yl}-1H-imidazole-4-carboxylate (D41)

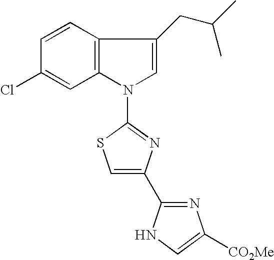

A mixture of 2-[6-chloro-3-isobutyl-1H-indol-1-yl]-N-hydroxy-1,3-thiazole-4-carboximidamide (980 mg, 2.81 mmol; may be prepared as described in D40) and methyl propiolate (236 mg, 2.81 mmol) in methanol (10 ml) was stirred and refluxed for 16 hours. The solution was cooled, evaporated and the residue dissolved in diphenyl ether (4 ml) and stirred and heated at 190° C. for 2 hours. The solution was cooled, poured into hexane and the solvent decanted. The residue was purified on a Biotage column eluting with 1:1 ethyl acetate/hexane and triturated with ether to give the title compound as a light brown solid (401 mg).

LCMS: Rt=3.76 min, [MH⁺] 415.19, 417.18.

### Description 42

### 2-(6-Chloro-3-isobutyl-1H-indol-1-yl)-N-[(1E)-(dimethylamino)methylene]-1,3-thiazole-4-carboxamide (D42)

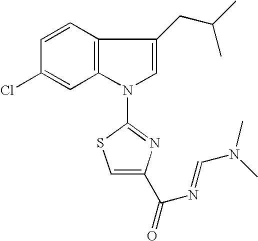

2-(6-Chloro-3-isobutyl-1H-indol-1-yl)-1,3-thiazole-4-carboxamide (190 mg, 0.57 mmol; may be prepared as described in E30) and dimethylformamide dimethylacetal (2 ml) were heated at 120° C. for 3 hrs; solution formed on heating. The solvent was evaporated and the residue triturated with ether to give the title compound (160 mg) as a yellow solid.

LCMS: Rt=3.37 min [M+H] 389.2.

### Description 43

### 2-(6-Chloro-3-isobutyl-1H-indol-1-yl)-N-[(1E)-1-(dimethylamino)ethylidene]-1,3-thiazole-4-carboxamide (D43)

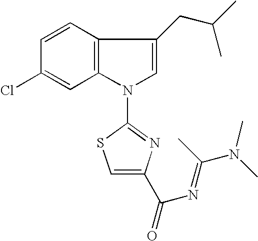

2-(6-Chloro-3-isobutyl-1H-indol-1-yl)-1,3-thiazole-4-carboxamide (333 mg, 1 mmol; may be prepared as described in E30) dissolved in dimethylacetamide dimethylacetal (5 ml) was heated under reflux for 2 hrs. The solvent was evaporated and the residue purified by flash chromatography on silica gel eluting with 1% methanol/dichloromethane to give the title compound (260 mg).

LCMS: Rt=3.02 min [M+H] 403.1.

### Example 1

### N-{2-[6-Chloro-3-isobutyl-1H-indol-1-yl]-1,3-thiazol-4-yl}-2-phenylacetamide (E1)

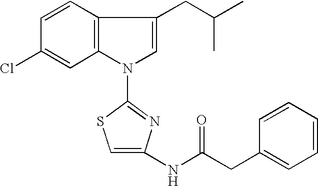

2-[6-Chloro-3-isobutyl-1H-indol-1-yl]-1,3-thiazol-4-amine hydrochloride (88 mg, 0.26 mmol; may be prepared as described in D31) was dissolved in DCM (5 ml), Et₃N (43 μl, 0.313 mmol) and phenylacetyl chloride (37.6 μl, 0.286 mmol) were added. The reaction mixture was stirred at room temperature overnight, dilute with water and extracted with DCM (×3). The combined organic phase was dried (MgSO₄) and evaporated. The residue was chromatographed using 10% of ethyl acetate in hexane to give the title compound as yellow solid (51 mg).

LCMS: Rt=4.34 min, [MH⁺] 424.2, 426.2, [MH⁺] 422.2, 424.4.

### Examples 2-4 (E2-E4)

The following compounds were prepared using an analogous method to that described in E1:

| Name | Structure | Data |
|---|---|---|
| N-{2-[6-Chloro-3- isobutyl-1H-indol-1-yl]- 1,3-thiazol-4- yl}benzamide (E2) | 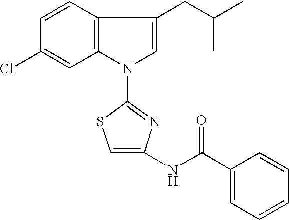 | LCMS Rt = 4.41 min [MH]⁺ 410.2, 412.1, [MH]⁻ 408.2, 410.2 |
| N-{2-[6-Chloro-3- isobutyl-1H-indol-1-yl]- 1,3-thiazol-4- yl}cyclopentanecarboxamide (E3) | 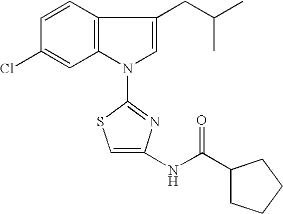 | LCMS Rt = 4.27 min [MH]⁺ 402.2, 404.2, [MH]⁻ 400.2, 402.2 |
| N-{2-[6-Chloro-3- isobutyl-1H-indoi-1-yl]- 1,3-thiazol-4-yl}-2,2- dimethylpropanamide (E4) | 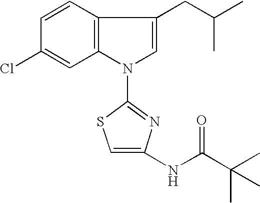 | LCMS Rt = 4.22 min [MH]⁺ 390.3, 392.3 [MH]⁻ 388.25, 391.3 |

### Example 5

### N-[2-(6-Chloro-3-isobutyl-1H-indol-1-yl)-1,3-thiazol-4-yl]-2,6-difluorobenzamide (E5)

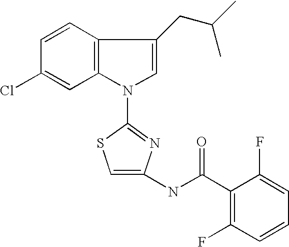

Oxalyl chloride (0.016 ml) was added to a solution of 2,6-difluorobenzoic acid (22 mg, 0.14 mmol) in dichloromethane (3 ml) and one drop of dimethylformamide, stirred at room temperature for 30 mins and evaporated to dryness. The residue was dissolved in dichloromethane (3 ml) and added to a solution of 2-[6-chloro-3-isobutyl-1H-indol-1-yl]-1,3-thiazol-4-amine hydrochloride (40 mg, 0.12 mmol; may be prepared as described in D31) in dichloromethane (2 ml) and triethylamine (0.033 ml, 0.24 mmol). The solution was stirred at room temperature for 1 hr, washed with 2M hydrochloric acid, saturated sodium bicarbonate solution, dried, evaporated and purified by MDAP to give the title compound (4 mg) as a white solid.

LCMS: Rt=4.22 min [M+H] 446.2.

### Example 6

### N-[2-(6-Chloro-3-isobutyl-1H-indol-1-yl)-1,3-thiazol-4-yl]nicotinamide (E6)

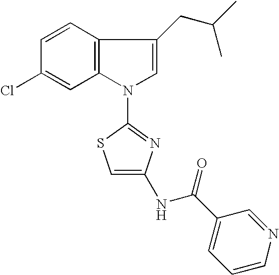

A solution of 2-[6-chloro-3-isobutyl-1H-indol-1-yl]-1,3-thiazol-4-amine hydrochloride (50 mg, 0.15 mmol; may be prepared as described in D31) in dichloromethane (3 ml) was treated with triethylamine (0.021 ml, 0.15 mmol) and nicotinoyl chloride hydrochloride (27 mg, 0.15 mmol) and stirred at room temperature for 1 hr. The solution was washed with saturated sodium bicarbonate solution, dried, evaporated and purified by MDAP to give the title compound (6 mg) as a yellow solid.

LCMS: Rt=4.05 min [M+H] 411.2.

### Examples 7-8 (E7-E8)

The following examples were made by an analogous method to that described in E6:

| Name | Structure | Data |
|---|---|---|
| N-{2-[6-Chloro-3- isobutyl-1H-indol-1-yl]- 1,3-thiazol-4-yl}-4- (methyloxy)benzamide (E7) | 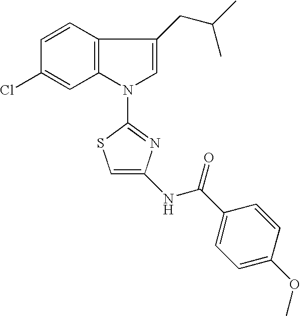 | LCMS Rt = 4.36 min [MH]⁺ 440.1 |
| N-{2-[6-Chloro-3- isobutyl-1H-indol-1-yl]- 1,3-thiazol-4-yl}-4- cyanobenzamide (E8) | 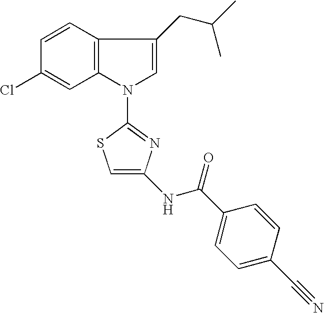 | LCMS Rt = 4.30 min [MH]⁺ 435.1 |

### Example 9

### N-[2-(6-Chloro-3-isobutyl-1H-indol-1-yl)-1,3-thiazol-4-yl]-4-(hydroxymethyl)benzamide (E9)

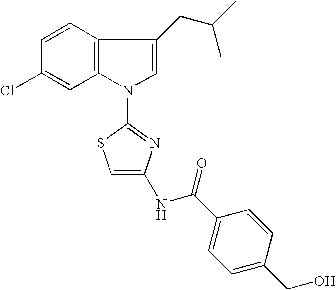

4-({[2-(6-Chloro-3-isobutyl-1H-indol-1-yl)-1,3-thiazol-4-yl]amino}carbonyl)benzyl acetate (20 mg, 0.04 mmol; may be prepared as described in D32) was dissolved in ethanol (1 ml), treated with 2M sodium hydroxide solution and stirred at room temperature for 1 hr. The solvent was evaporated, the residue dissolved in 2M hydrochloric acid, extracted with dichloromethane, dried, evaporated and triturated with ethyl acetate/hexane to give the title compound (14 mg) as a yellow solid.

LC/MS: Rt=3.98 min [M+H] 440.1.

### Example 10

### N-[2-(6-Chloro-3-isobutyl-1H-indol-1-yl)-1,3-thiazol-4-yl]-N′-propylurea (E10)

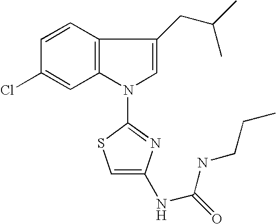

2-(6-Chloro-3-isobutyl-1H-indol-1-yl)-1,3-thiazol-4-amine hydrochloride (50 mg, 0.15 mmol; may be prepared as described in D31) dissolved in dichloromethane (3 ml) was treated with triethylamine (0.021 ml, 0.15 mmol) and n-propyl isocyanate (0.014 ml, 0.15 mmol) and stirred at room temperature for 1 hr. The solution was washed with saturated sodium bicarbonate solution, dried, evaporated and purified by MDAP to give the title compound (6 mg).

LCMS: Rt=4.1 min [M+H] 391.2.

### Example 11

### 6-Chloro-3-isobutyl-1-[4-(1-pyrrolidinylcarbonyl)-1,3-thiazol-2-yl]-1H-indole (E11)

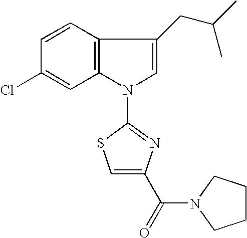

A mixture of 2-[6-chloro-3-isobutyl-1H-indol-1-yl]-1,3-thiazole-4-carboxylic acid (50 mg, 0.15 mmol; may be prepared as described in D29), 1-hydroxybenzotriazole (31 mg, 0.2 mmol), EDAC (38 mg, 0.2 mmol) and pyrrolidine (0.2 mmol) in dichloromethane (3 ml) was stirred at room temperature for 20 hours. The resulting solution was diluted with ether, washed with 2M hydrochloric acid and saturated sodium bicarbonate then dried (magnesium sulphate), evaporated and triturated with hexane to give the title compound as a white solid.

LCMS: Rt=4.00 min, [MH⁺] 388.26, 390.23.

### Examples 12-29 (E12-E29)

The following compounds were prepared by coupling an acid to the appropriate amine using an analogous method to that described in E11 triturating with hexane, ether or a mixture of hexane and ether, or purifying on MDAP as necessary:

| Name | Structure | Data |
|---|---|---|
| 2-[6-Bromo-3-isobutyl-1H- indol-1-yl]-N-[4- (hydroxymethyl)phenyl]- 1,3-thiazole-4-carboxamide (E12) | 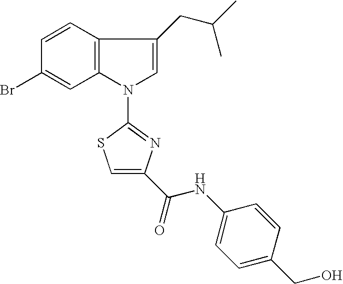 | LCMS Rt = 3.94 min [MH]⁺ 484.22 |
| N-(2-Aminophenyl)-2-[6- chloro-3-isopropyl-1H- indol-1-yl]-1,3-thiazole-4- carboxamide (E13) | 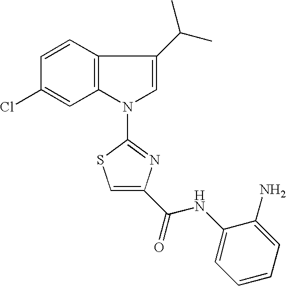 | LCMS Rt = 3.85 min [MH]⁺ 411.19, 413.20 |
| N-(2-Aminophenyl)-2-[6- chloro-3-isobutyl-1H-indol- 1-yl]-1,3-thiazole-4- carboxamide (E14) | 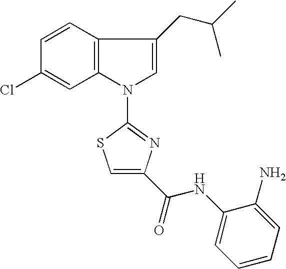 | LCMS Rt = 3.84 min [MH]⁺ 425.23, 427.23 |
| N-(2-Aminophenyl)-2-[6- chloro-3-isobutyl-1H-indol- 1-yl]-1,3-oxazole-4- carboxamide (E15) | 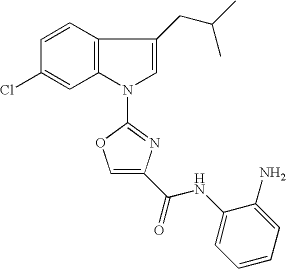 | LCMS Rt = 3.86 min [MH]⁺ 409.28, 411.24 |
| N-(2-Aminophenyl)-2-[6- chloro-3-(3,3- dimethylbutyl)-1H-indol-1- yl]-1,3-thiazole-4- carboxamide (E16) | 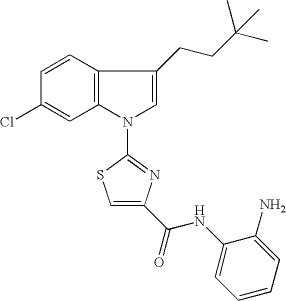 | LCMS Rt = 4.32 min [MH]⁺ 453.25, 455.23 |
| N-(2-Aminophenyl)-2-[6- bromo-3-isobutyl-1H-indol- 1-yl]-1,3-thiazole-4- carboxamide (E17) | 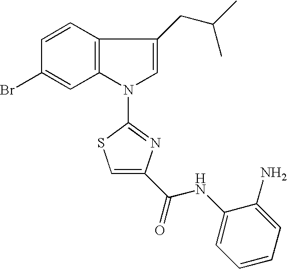 | LCMS Rt = 4.00 min [MH]⁺ 472.13 |
| N-(2-Aminophenyl)-2-[6- chloro-3-isobutyl-1H- indazol-1-yl]-1,3-thiazole-4- carboxamide (E18) | 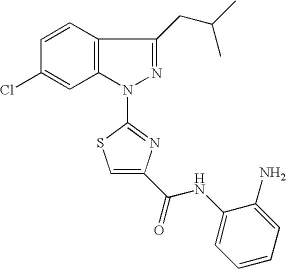 | LCMS Rt = 3.86 min [MH]⁺ 428.21 |
| N-(2-Aminophenyl)-2-{[6- chloro-3-isobutyl-1H-indol- 1-yl]methyl}-1,3-thiazole-4- carboxamide (E19) | 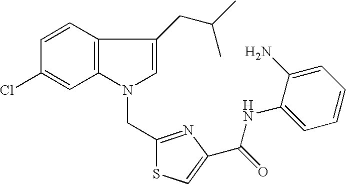 | LCMS Rt = 3.86 min [MH]⁺ 441.17 |
| 2-[6-Chloro-3-isobutyl-1H- indol-1-yl]-N-[2- (hydroxymethyl)phenyl]- 1,3-thiazole-4-carboxamide (E20) | 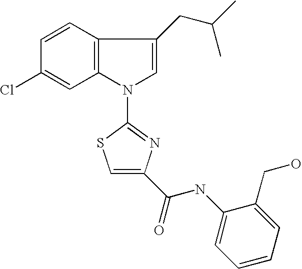 | LCMS Rt = 4.18 min [MH]⁺ 440.1, |
| 2-[6-Chloro-3-isobutyl-1H- indol-1-yl]-N-[4- (hydroxymethyl)phenyl]- 1,3-thiazole-4-carboxamide (E21) | 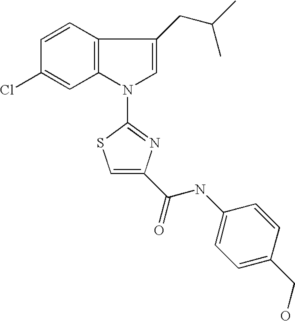 | LCMS Rt = 3.93 min |
| 2-[6-Chloro-3-isobutyl-1H- indol-1-yl]-N-3-pyridinyl- 1,3-thiazole-4-carboxamide (E22) | 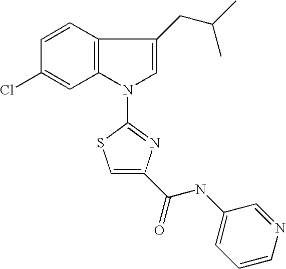 | LCMS Rt = 3.82 min [MH]⁺ 411.1 |
| 2-[6-Chloro-3-isobutyl-1H- indol-1-yl]N-phenyl-1,3- thiazoie-4-carboxamide (E23) | 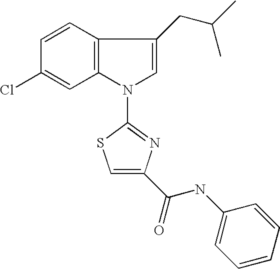 | LCMS Rt = 4.43 min [MH]⁺ 472.3 |
| 2-[6-Chloro-3-isobutyl-1H- indol-1-yl]-N-[3- indol-1-yl]N-[3- (hydroxymethyl)phenyl]- 1,3-thiazole-4-carboxamide (E24) | 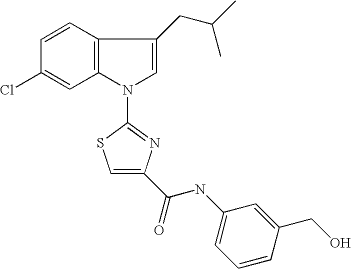 | LCMS Rt = 4.01 min [MH]⁻ 438.0 |
| 2-[6-Chloro-3-isobutyl-1H- indol-1-yl]-N-4-pyridinyl- 1,3-thiazole-4-carboxamide (E25) | 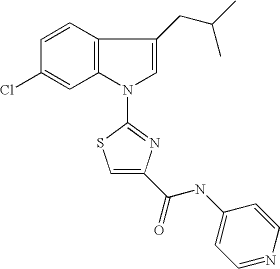 | LCMS Rt = 2.83 min [MH]⁺ 411.1 |
| 2-[6-Chloro-3-isobutyl-1H- indol-1-yl]-N-(3- hydroxypropyl)-1,3- thiazole-4-carboxamide (E26) | 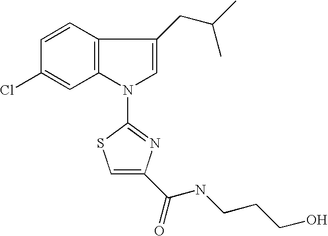 | LCMS Rt = 3.57 min [MH]⁺ 392.1 |
| 2-[6-Chloro-3-isobutyl-1H- indol-1-yl]-N-2-pyridinyl- 1,3-thiazole-4-carboxamide (E27) | 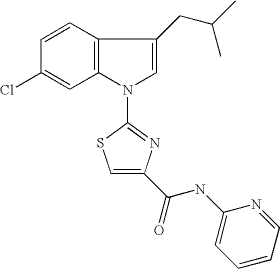 | LCMS Rt = 4.30 min [MH]⁺ 411.1 |
| N-(2-Aminophenyl)-2-[3- isobutyl-6-(trifluoromethyl)- 1H-indol-1-yl]-1,3-thiazole- 4-carboxamide (E28) | 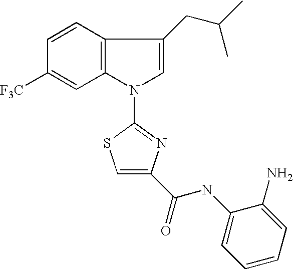 | LCMS Rt = 3.98 min [MH]⁺ 459.0 |
| 2-[6-Chloro-3-isobutyl-1H- indol-1-yl]-N-(4- fluorophenyl)-1,3-thiazole- 4-carboxamide (E29) | 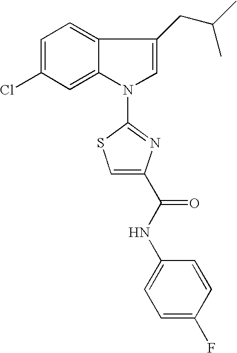 | LCMS Rt = 4.44 min [MH]⁺ 428.1 |

### Example 30

### 2-[6-Chloro-3-isobutyl-1H-indol-1-yl]-1,3-thiazole-4-carboxamide (E30)

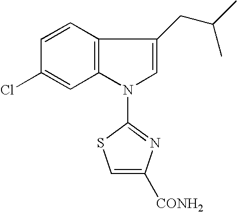

### Preparation A

A mixture of 2-[6-chloro-3-isobutyl-1H-indol-1-yl]-1,3-thiazole-4-carboxylic acid (50 mg, 0.15 mmol; may be prepared as described in D29), hydroxybenzotriazole ammonium salt (28 mg, 0.18 mmol), EDAC (38 mg, 0.2 mmol) and triethylamine (40 mg, 0.4 mmol) in dichloromethane (3 ml) was stirred at room temperature for 24 hours. The resulting solution was diluted with ethyl acetate, washed with saturated sodium bicarbonate, dried (magnesium sulphate), evaporated and triturated with ether to give the title compound as a white solid (31 mg).

LCMS: Rt=3.42 min, [MH⁺] 334.21, 336.20.

### Preparation B

2-(6-Chloro-3-isobutyl-1H-indol-1-yl)-1,3-thiazole-4-carboxylic acid (1 g, 3 mmol; may be prepared as described in D29), HOBt ammonium salt (608 mg, 6 mmol), EDAC (690 mg, 3.6 mmol) and N-methyl morpholine (0.66 ml, 6 mmol) were dissolved in dichloromethane (5 ml) and stirred at room temperature over a weekend. The solution was washed with saturated sodium bicarbonate solution, dried, evaporated and triturated with hexane to give the title compound (580 mg)

LCMS: Rt=3.63 min [M+H] 334.1

### Examples 31-32 (E31-E32)

The following compounds were prepared in an analogous manner to that described in E30:

| Name | Structure | Data |
|---|---|---|
| 2-[6-Chloro-3-(3,3- dimethylbutyl)-1H-indol-1- yl]-1,3-thiazole-4- carboxamide (E31) | 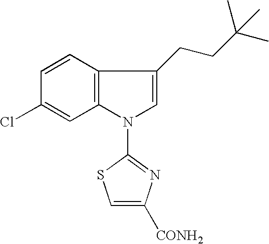 | LC/MS Rt = 4.00 min [MH]⁺362.18, 364.20 |
| 2-[6-Bromo-3-isobutyl-1H- indol-1-yl]-1,3-thiazole-4- carboxamide (E32) | 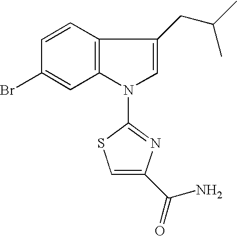 | LC/MS Rt = 3.62 min [MH]⁺ 378.11 |

### Example 33

### 2-[6-Chloro-3-isobutyl-1H-indol-1-yl]-N-(2,6-difluorophenyl)-1,3-thiazole-4-carboxamide (E33)

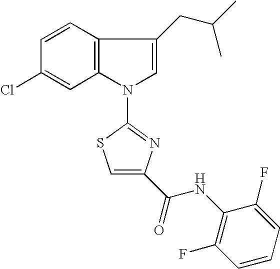

Oxalyl chloride (0.1 ml) was added to a suspension of 2-[6-chloro-3-isobutyl-1H-indol-1-yl]-1,3-thiazole-4-carboxylic acid (50 mg, 0.15 mmol; may be prepared as described in D29) and one drop of dimethylformamide in dichloromethane (5 ml) and stirred at room temperature for 2 hours. The resulting solution was evaporated, dissolved in dichloromethane (1 ml) and added to a solution of 2,6-difluoroaniline (26 mg, 0.2 mmol) in 1:1 dichloromethane/pyridine (2 ml). Left for 2 hours then diluted with ethyl acetate and washed with 2M hydrochloric acid and saturated sodium bicarbonate. The organic phase was dried (magnesium sulphate) evaporated, purified on a Biotage column eluting with 1:4 ethyl acetate/hexane. The product was triturated with 1:1 ether/hexane to give the title compound as a white solid (44 mg).

LCMS: Rt=4.15 min, [MH⁺] 446.18, 448.18

### Example 34

### 2-{[6-Chloro-3-isobutyl-1H-indol-1-yl]methyl}-1,3-thiazole-4-carboxamide (E34)

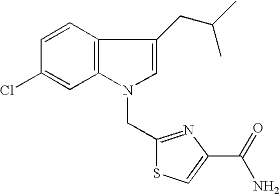

2-{[6-Chloro-3-isobutyl-1H-indol-1-yl]methyl}-1,3-thiazole-4-carboxylic acid (170 mg, 0.49 mmol; may be prepared as described in D26) in dichloromethane (10 ml) containing one drop of DMF were treated with oxalyl chloride (0.4 ml) and stirred for 90 minutes. The solution was evaporated to dryness, dissolved in dichloromethane (6 ml) and 2 ml of the solution were added to pyridine (1 ml) followed by concentrated aqueous ammonia (0.5 ml) with stirring. After 30 minutes the mixture was diluted with dichloromethane and washed with 2M hydrochloric acid then dried with magnesium sulphate, evaporated to dryness and purified on a Biotage silica column eluting with ethyl acetate/hexane (1:1) to give the title compound as a solid after trituration with hexane.

LCMS: Rt=3.31 min, [MH⁺] 348.23, 350.22.

### Example 35

### 2-{2-[6-Chloro-3-isobutyl-1H-indol-1-yl]-1,3-thiazol-4-yl}-1H-benzimidazole hydrochloride (E35)

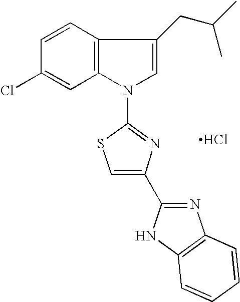

N-(2-Aminophenyl)-2-[6-chloro-3-isobutyl-1H-indol-1-yl]-1,3-thiazole-4-carboxamide (101 mg, 0.24 mmol; may be prepared as described in E14) in acetic acid (5 ml) was stirred and refluxed for one hour then cooled and evaporated. The residue was dissolved in ethyl acetate, washed with saturated sodium bicarbonate, dried (magnesium sulphate) and evaporated. The residue was dissolved in dichloromethane, 1M hydrogen chloride in ether (1 ml) added and the solvent evaporated. The residue was triturated with ether to give the title compound as a pale pink solid (66 mg).

LCMS: Rt=3.53 min, [MH⁺] 407.22, 409.22.

### Examples 36-40 (E36-E40)

The following compounds were prepared using an analogous method to that described in E35:

| Name | Structure | Data |
|---|---|---|
| 2-{2-[6-Chloro-3-isobutyl-1H- indol-1-yl]-1,3-oxazol-4-yl}-1H- benzimidazole hydrochloride (E36) | 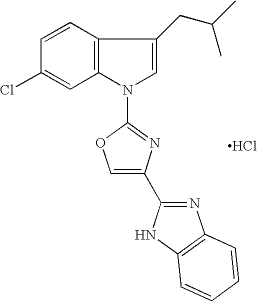 | LC/MS Rt = 3.79 min [MH]⁺ 391.28, 393.27 |
| 2-{2-[6-Chloro-3-(3,3- dimethylbutyl)-1H-indol-1-yl]- 1,3-thiazol-4-yl}-1H- benzimidazole hydrochloride (E37) | 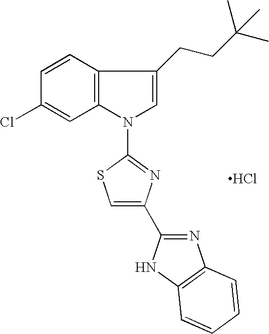 | LC/MS Rt = 4.24 min [MH]⁺ 435.19, 437.22 |
| 2-{2-[6-Bromo-3-isobutyl-1H- indol-1-yl]-1,3-thiazol-4-yl}-1H- benzimidazole hydrochloride (E38) | 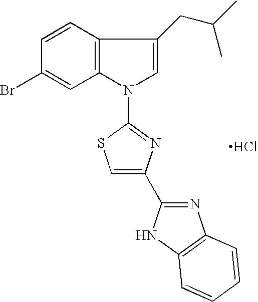 | LC/MS Rt = 3.75 min [MH]⁺ 453.13 |
| 1-[4-(1H-Benzimidazol-2-yl)- 1,3-thiazol-2-yl]-6-chloro-3- isobutyl-1H-indazole hydrochloride (E39) | 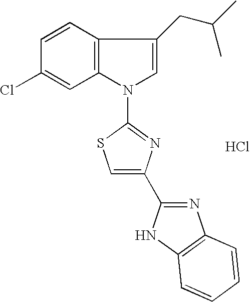 | LC/MS Rt = 3.55 min [MH]⁺ 408.22, 410.21 |
| 2-(2-{[6-Chloro-3-isobutyl-1H- indol-1-yl]methyl}-1,3-thiazol- 4-yl)-1H-benzimidazole hydrochloride (E40) | 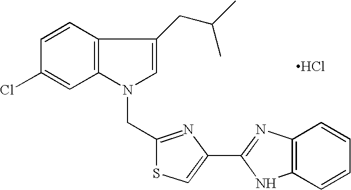 | LC/MS Rt = 3.21 min [MH]⁺ 421.24, 423.23 |

### Example 41

### 2-{2-[3-Isobutyl-6-(trifluoromethyl)-1H-indol-1-yl]-1,3-thiazol-4-yl}-1H-benzimidazole (E41)

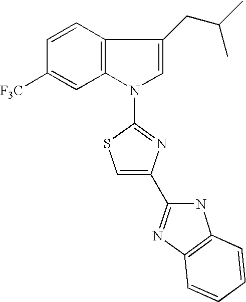

N-(2-Aminophenyl)-2-[3-isobutyl-6-(trifluoromethyl)-1H-indol-1-yl]-1,3-thiazole-4-carboxamide (30 mg, 0.0068 mmol; may be prepared as described in E28) dissolved in acetic acid (1 ml) was heated at 110° C. for 2 hours. The solution was diluted with water and extracted with ethyl acetate. The organic layer was washed with water, dried (magnesium sulphate), evaporated and triturated with ether/hexane to give the title compound (6 mg) as a white solid.

LCMS: Rt=3.82 min, [MH]⁺ 441.1.

### Example 42

### 2-{2-[6-Chloro-3-isobutyl-1H-indol-1-yl]-1,3-thiazol-4-yl}-1H-imidazo[4,5-b]pyridine hydrochloride (E42)

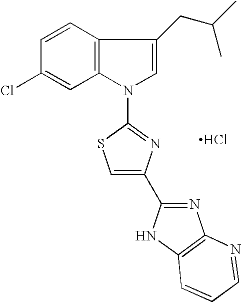

A mixture of 2-[6-chloro-3-isobutyl-1H-indol-1-yl]-1,3-thiazole-4-carboxylic acid (134 mg, 0.4 mmol; may be prepared as described in D29), 1-hydroxybenzotriazole (67 mg, 0.44 mmol), EDAC (115 mg, 0.6 mmol) and 2,3-diaminopyridine (55 mg, 0.5 mmol) in dichloromethane (3 ml) and dimethylformamide (1 ml) was stirred at room temperature for 24 hours. The resulting solution was diluted with ethyl acetate, washed twice with saturated sodium bicarbonate and twice with water then dried (magnesium sulphate), evaporated, triturated with ether and filtered. The resulting off-white solid was stirred and refluxed in propionic acid (3 ml) for 6 hours and the solution cooled, diluted with ethyl acetate and washed three times with saturated sodium bicarbonate. The organic phase was dried (magnesium sulphate), evaporated, triturated with dichloromethane and the solid filtered off. It was dissolved in dichloromethane/methanol and 1M hydrogen chloride in ether (1 ml) added. After evaporation of the solvent the residue was triturated with ether to give the title compound as an off-white solid (30 mg).

LCMS: Rt=3.61 min, [MH⁺] 408.18, 410.21.

### Examples 43-44 (E43-E44)

The following compounds were prepared in an analogous manner to that described in E42:

| Name | Structure | Data |
|---|---|---|
| 2-{2-[6-Chloro-3-isobutyl-1H- indol-1-yl]-1,3-thiazol-4-yl}- 1H-imidazo[4,5-c]pyridine hydrochloride (E43) | 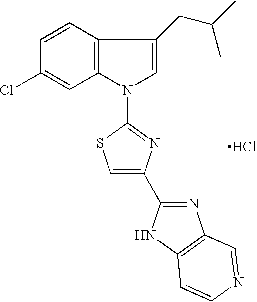 | LC/MS Rt = 2.32 min [MH]⁺ 408.22, 410.21 |
| Methyl 2-{2-[6-chloro-3- isobutyl-1H-indol-1-yl]-1,3- thiazol-4-yl}-1H- benzimidazole-5-carboxylate (E44) | 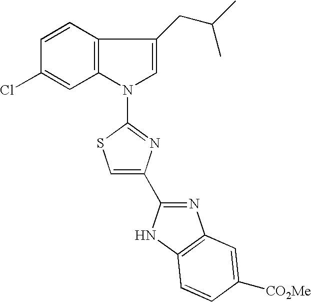 | LC/MS Rt = 4.21 min [MH]⁺ 465.23, 467.22 |

### Example 45

### (2-{2-[6-Chloro-3-isobutyl-1H-indol-1-yl]-1,3-thiazol-4-yl}-1H-benzimidazol-5-yl)methanol hydrochloride (E45)

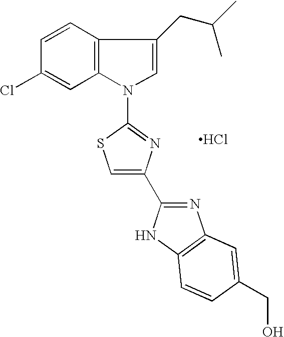

1M Lithium aluminium hydride (3 ml) was added to a solution of methyl 2-{2-[6-chloro-3-isobutyl-1H-indol-1-yl]-1,3-thiazol-4-yl}-1H-benzimidazole-5-carboxylate (1.08 g, 2.32 mmol; may be prepared as described in E44) in dry THF (20 ml) under argon and stirred for one hour. 2M sodium hydroxide was added carefully followed by ethyl acetate and the organic phase was dried (magnesium sulphate), evaporated, triturated with ether and filtered to give an off-white solid (810 mg). 45 mg of this solid were dissolved in dichloromethane and 1M hydrogen chloride in ether (1 ml) added. After evaporation of the solvent the residue was triturated with ether to give the title compound as a white solid (30 mg).

LCMS: Rt=3.16 min, [MH⁺] 437.18, 439.18.

### Example 46

### (2-{2-[6-Chloro-3-isobutyl-1H-indol-1-yl]-1,3-thiazol-4-yl}-1H-benzimidazol-4-yl)methanol (E46)

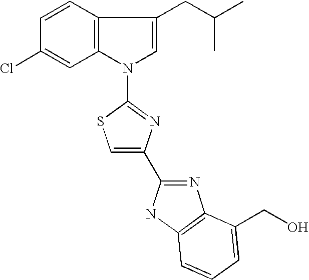

1M Lithium aluminium hydride (1.3 ml) was added at −10° C. to a solution of methyl 2-{2-[6-chloro-3-isobutyl-1H-indol-1-yl]-1,3-thiazol-4-yl}-1H-benzimidazole-4-carboxylate (550 mg, 1.18 mmol; may be prepared as described in D33) in dry THF (8 ml) under argon, the solution was stirred for 2 hr at room temperature. More 1M Lithium aluminium hydride (0.3 ml) was added and mixture was stirred for 1 hr. 2M sodium hydroxide was added carefully followed by ethyl acetate and the organic phase was dried and evaporated. The residue was purified on a silica column using ethyl acetate; after evaporation of the solvent the residue was triturated with ether to give the title compound as a pale yellow solid (130 mg).

LCMS: Rt=3.33 min, [MH⁺] 437.1, 439.1, [MH⁻] 435.2, 437.1.

### Example 47

### 6-Chloro-1-[4-(1H-imidazol-2-yl)-1,3-thiazol-2-yl]-3-isobutyl-1H-indole (E47)

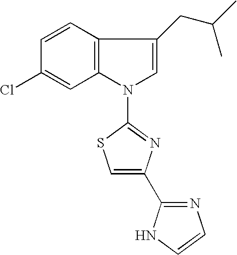

40% Glyoxal (0.18 ml) was added to a stirred solution of 2-[6-chloro-3-isobutyl-1H-indol-1-yl]-1,3-thiazole-4-carbaldehyde (128 mg, 0.4 mmol; may be prepared as described in D35) in 2:1 THF/ethanol followed by concentrated aqueous ammonia (0.27 ml). The mixture was stirred for 20 hours at room temperature then diluted with ethyl acetate and washed twice with water. The organic phase was dried (magnesium sulphate), evaporated and purified on a Biotage column eluting with ethyl acetate/hexane (1:1). The product was triturated with ether to give the title compound as a white solid (34 mg).

LCMS: Rt=2.35 min, [MH⁺] 357.19, 359.19.

### Example 48

### [(2-{2-[6-Chloro-3-isobutyl-1H-indol-1-yl]-1,3-thiazol-4-yl}-1H-benzimidazol-5-yl)methyl]methylamine dihydrochloride (E48)

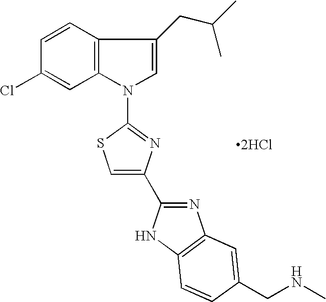

Sodium triacetoxyborohydride (84 mg, 0.4 mmol) was added to a stirred solution of 2-{2-[6-chloro-3-isobutyl-1H-indol-1-yl]-1,3-thiazol-4-yl}-1H-benzimidazole-5-carbaldehyde (87 mg, 0.2 mmol; may be prepared as described in D36) and 33% ethanolic methylamine (0.037 ml, 0.4 mmol) in THF (4 ml). The mixture was stirred for 18 hours and sodium borohydride (15 mg, 0.4 mmol) added and stirred for a further 4 hours. The solution was diluted with dichloromethane/water and the organic phase dried (magnesium sulphate), evaporated and purified on a biotage column eluting with methanol/dichloromethane. Dissolved in dichloromethane and 1M hydrogen chloride in ether (1 ml) added. After evaporation of the solvent the residue was triturated with ether to give the title compound as a white solid (21 mg).

LCMS: Rt=2.21 min, [MH⁻] 448.19, 450.20.

### Examples 49-51 (E49-E51)

The following compounds were prepared in an analogous manner to that described in E48 except that sodium borohydride was not added to the mixture:

| Name | Structure | Data |
|---|---|---|
| [(2-{2-[6-Chloro-3-isobutyl-1H- indol-1-yl]-1,3-thiazol-4-yl}-1H- benzimidazol-5- yl)methyl]dimethylamine dihydrochloride (E49) | 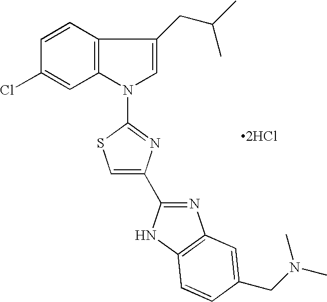 | LCMS Rt = 2.25 min [MH]⁻ 462.21, 464.20 |
| 2-{2-[6-Chloro-3-isobutyl-1H- indol-1-yl]-1,3-thiazol-4-yl}-5-(1- pyrrolidinylmethyl)-1H- benzimidazole dihydrochloride (E50) | 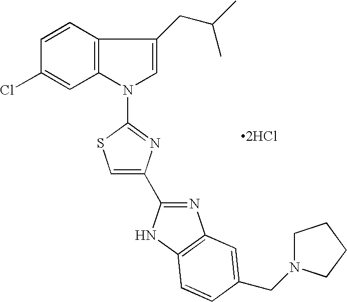 | LCMS Rt = 2.62 min [MH]⁺ 488.16, 490.14 |
| 2-{2-[6-Chloro-3-isobutyl-1H- indol-1-yl]-1,3-thiazol-4-yl}-5-(1- piperidinylmethyl)-1H- benzimidazole dihydrochloride (E51) | 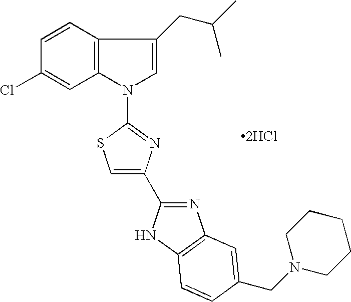 | LCMS Rt = 2.34 min [MH]⁺ 502.24, 504.20 |

### Example 52

### 6-Chloro-3-isobutyl-1-[4-(4-phenyl-1H-imidazol-2-yl)-1,3-thiazol-2-yl]-1H-indole (E52)

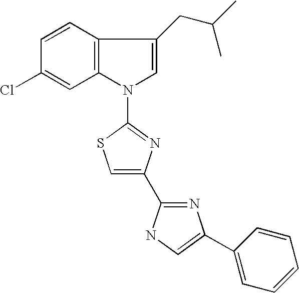

A solution of 1-bromo-3-phenyl-2-propanone (71 mg, 0.33 mmol) in THF (0.4 ml) was added to a refluxing solution of 2-[6-chloro-3-isobutyl-1H-indol-1-yl]-1,3-thiazole-4-carboximidamide (100 mg, 0.3 mmol; may be prepared as described in D39) and potassium bicarbonate (120 mg, 1.2 mmol) in THF/water (4:1, 2 ml). The resulting mixture was stirred and refluxed for 4 hours then cooled, diluted with ethyl acetate/water and the organic phase was dried (magnesium sulphate), evaporated and purified on a Biotage column eluting with ethyl acetate/hexane (1:4) then triturated with ether to give the title compound as a solid (15 mg).

LCMS: Rt=3.65 min, [MH⁺] 433.2, 435.2.

### Example 53

### (2-{2-[6-Chloro-3-isobutyl-1H-indol-1-yl]-1,3-thiazol-4-yl}-1H-imidazol-4-yl)methanol (E53)

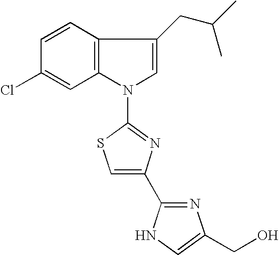

1M Lithium aluminium hydride (1.5 ml) was added to a solution of methyl 2-{2-[6-chloro-3-isobutyl-1H-indol-1-yl]-1,3-thiazol-4-yl}-1H-imidazole-4-carboxylate (395 mg, 0.95 mmol; may be prepared as described in D41) in dry THF (8 ml) under argon and stirred for one hour. 2M sodium hydroxide was added carefully followed by ethyl acetate and the organic phase was dried (magnesium sulphate), evaporated and purified on a Biotage column eluting with ethyl acetate to remove impurities and 1:19 methanol/ethyl acetate to elute the product as a white solid (157 mg).

LCMS: Rt=2.58 min, [MH⁺] 387.22, 389.29.

### Example 54

### 3-[2-(6-Chloro-3-isobutyl-1H-indol-1-yl)-1,3-thiazol-4-yl]-4H-1,2,4-triazole (E54)

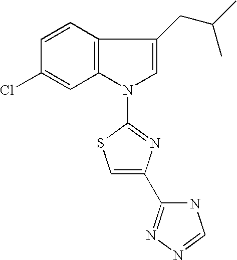

2-(6-Chloro-3-isobutyl-1H-indol-1-yl)-N-[(1E)-(dimethylamino)methylene]-1,3-thiazole-4-carboxamide (may be prepared as described in D42) was dissolved in glacial acetic acid (4 ml), treated with hydrazine hydrate (0.021 ml, 0.44 mmol) and heated at 100° C. for 30 mins. The solvent was evaporated, azeotroped with toluene and triturated with ether to give the title compound (76 mg) as an off white solid.

LCMS: Rt=3.56 [M+H] 358.2.

### Example 55

### 3-[2-(6-Chloro-3-isobutyl-1H-indol-1-yl)-1,3-thiazol-4-yl]-5-methyl-4H-1,2,4-triazole (E55)

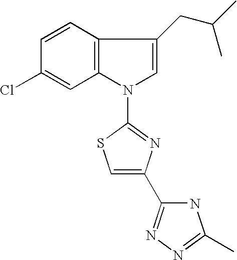

2-(6-Chloro-3-isobutyl-1H-indol-1-yl)-N-[(1E)-1-(dimethylamino)ethylidene]-1,3-thiazole-4-carboxamide (260 mg, 0.65 mmol; may be prepared as described in D43) was dissolved in glacial acetic acid (3 ml), treated with hydrazine hydrate (0.034 ml, 0.7 mmol) and heated at 100° C. for 30 mins. The solvent was evaporated, the residue triturated with ethyl acetate/hexane and the solid further purified by MDAP to give the title compound as a whitish solid (21 mg).

LCMS: Rt=3.62 min [M+H] 372.1.

### Example 56

### {4-[({2-[6-Chloro-3-(2-methylpropyl)-1H-indol-1-yl]-1,3-thiazol-4-yl}amino)carbonyl]phenyl}methyl acetate (E56) (Also D32)

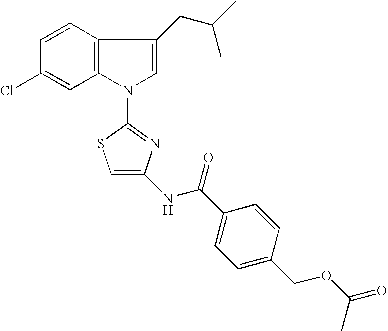

A solution of 4-acetoxymethyl benzoic acid (43 mg, 0.22 mmol) in DCM (5 ml) was treated with oxalyl chloride (29 uL, 0.33 mmol) followed by DMF (1 drop). Fizzing occurred. Stirred at room temperature for 30 minutes—LC/MS showed no acid remaining (quenched with MeOH). The solvents were evaporated, re-evaporated with toluene and dissolved in DCM (5 mL). Treated with 2-[6-chloro-3-isobutyl-1H-indol-1-yl]-1,3-thiazol-4-amine hydrochloride (68 mg, 0.2 mmol; may be prepared as described in D31) and triethylamine (61 μL, 0.44 mmol)—the solution turned yellow. Stirred at room temperature for 1 hr. Washed with 2M HCl, sat. NaHCO₃, dried and evaporated. Purified by flash chromatography eluting with 1:5 EtOAc/hexane to give the title compound (45 mg) as a yellow solid.

LCMS: Rt=4.33 min [M+H] 482.2.

It is to be understood that the present invention covers all combinations of particular and preferred subgroups described herein above.

### Assays for Determining Biological Activity

The compounds of formula (I) can be tested using the following assays to demonstrate their prostanoid antagonist or agonist activity in vitro and in vivo and their selectivity. Prostaglandin receptors that may be investigated are DP, EP₁, EP₂, EP₃, EP₄, FP, IP and TP.

### Biological Activity at EP₁ and EP₃ Receptors

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable (pCIN; BioTechniques 20 (1996): 102-110) vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine. The concentration of compound that elicits a half-maximal inhibition of the calcium mobilisation induced by PGE₂ (pIC₅₀) may then be estimated.

### Binding Assay for the Human Prostanoid EP₁ Receptor

Competition assay using [³H]-PGE2.

Compound potencies are determined using a radioligand binding assay. In this assay compound potencies are determined from their ability to compete with tritiated prostaglandin E₂ ([³H]-PGE₂) for binding to the human EP₁ receptor. This assay utilises Chinese hamster ovary-K1 (CHO-K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM indomethacin for 5 min. The cells are isolated by centrifugation at 250×g for 5 mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10 s burst at full setting), centrifuged at 48,000×g for 20 mins and the pellet containing the membrane fraction is washed (optional) three times by suspension and centrifugation at 48,000×g for 20 mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at −80° C. until required.

For the binding assay the cell membranes, competing compounds and [³H]-PGE₂ (3 nM final assay concentration) are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques to determine the concentration of compound producing 50% inhibition of specific binding (IC₅₀).

### RESULTS

The compounds of examples 1-13, 20-27, 29-43 and 45-56 were tested in the binding assay for the human prostanoid EP₁ receptor. The results are expressed as pIC₅₀ values. A pIC₅₀ is the negative logarithms of the IC₅₀. The results given are averages of a number of experiments. The compounds of examples 1-13, 20-27, 29-43 and 45-56 had a pIC₅₀ value 26. More particularly, the compounds of examples 3-4, 12-13, 20-21, 33, 35-45, 47 and 55 exhibited a pIC₅₀ value ≧7.

The compounds of examples 1, 3-12, 20-27, 29-41, 43 and 45-56 were tested in the human EP₁ calcium mobilisation assay. The results are expressed as functional pKᵢ values. A functional pKᵢ is the negative logarithms of the antagonist dissociation constant as determined in the human EP₁ calcium mobilisation assay. The results given are averages of a number of experiments. The compounds of examples 1, 3-12, 20-27, 29-41, 43 and 45-56 exhibited a functional pK₁ value ≧5.5. More particularly, the compounds of examples 5, 11-12, 20, 24, 26-27, 29, 30, 32-35, 38, 40-41, 43, 46 and 52 exhibited a functional pKᵢ value of ≧7.0.

The compounds of examples 1-13, 20, 22, 25-28, 30-40, 42, 45-56 were tested in the human EP₃ calcium mobilisation assay. The results are expressed as functional pKᵢ values. A functional pKᵢ is the negative logarithms of the antagonist dissociation constant as determined in the human EP₃ calcium mobilisation assay. The results given are averages of a number of experiments. The compounds of examples 6, 9, 10, 25, 33, 36, 45, 46, 54, 55 and 56 exhibited a functional pKᵢ value of >5.5. The remaining compounds tested exhibited a functional pKᵢ value of ≦5.5 or were inactive.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I): 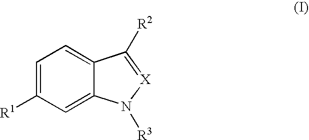
wherein
R¹ represents —CF₃, chlorine or bromine;
R² represents isopropyl, isobutyl or —(CH₂)₂-t-butyl;
X represents CH or N;
R³ represents a group of formula (i)-(iv): 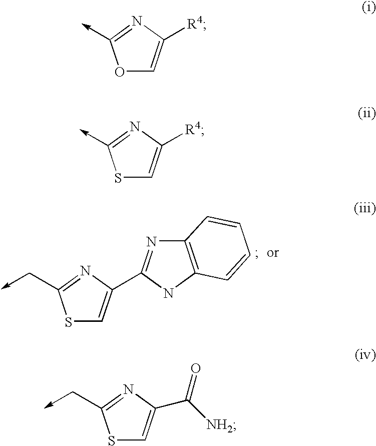
R⁴ represents —CO—NH—R⁵, —NH—CO—R⁶, —CO-pyrrolidinyl or a group of formula (v)-(viii): 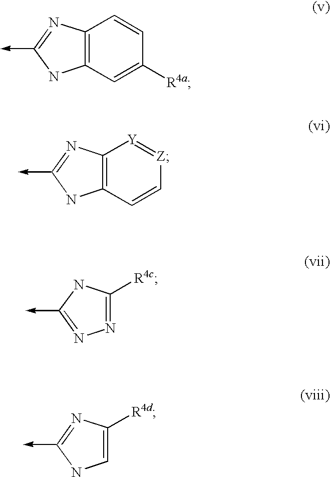
R^{4a} represents hydrogen, —CH₂OH or —CH₂—NR^{a}R^{b};
R^{4c} represents hydrogen or methyl;
R^{4d} represents hydrogen, —CH₂OH or optionally substituted phenyl;
R⁵ represents hydrogen, —(CH₂)₃—OH, pyridyl or optionally substituted phenyl;
R⁶ represents t-butyl, cyclopentyl, NR^{a}R^{b}, pyridyl or optionally substituted phenyl or benzyl;
R^{a} and R^{b} independently represent hydrogen or C₁₋₃ alkyl or R^{a} and R^{b} together with the nitrogen atom which they are attached form an N-pyrrolidinyl, N-piperidinyl or N-morpholinyl ring;
one of Y and Z represents CH and the other represents N;
such that when R² represents —(CH₂)₂-t-butyl, R³ represents a group of formula (iii) or (iv) and such that when R² represent isopropyl, R³ represents a group of formula (ii), R⁴ represents —CO—NH—R⁵ and R⁵ represents 2-aminophenyl;
or derivatives thereof.

2. (canceled)

3. A pharmaceutical composition comprising a compound according to claim 1 or a pharmaceutically acceptable derivative thereof together with a pharmaceutical carrier and/or excipient.

4-5. (canceled)

6. A method of treating a human or animal subject suffering from a condition which is mediated by the action of PGE₂ at EP₁ receptors which comprises administering to said subject an effective amount of a compound according to claim 1 or a pharmaceutically acceptable derivative thereof.

7. A method of treating a human or animal subject suffering from a pain, or an inflammatory, immunological, bone, neurodegenerative or renal disorder, which method comprises administering to said subject an effective amount of a compound according to claim 1 or a pharmaceutically acceptable derivative thereof.

8. A method of treating a human or animal subject suffering from inflammatory pain, neuropathic pain or visceral pain which method comprises administering to said subject an effective amount of a compound according to claim 1 or a pharmaceutically acceptable derivative thereof.

9-11. (canceled)
